(19) United States
(12) Patent Application Publication
(10) Pub. No.: US 2003/0170248 A1
(43) Pub. Date: Sep. 11, 2003

(54) HUMANIZED MONOCLONAL ANTIBODIES THAT PROTECT AGAINST SHIGA TOXIN INDUCED DISEASE

(76) Inventors: JEFFREY R. STINSON, DAVIE, FL (US); HING WONG, WESTON, FL (US); ALISON D. OBRIEN, BETHESDA, MD (US); CLARE K. SCHMITT, BETHESDA, MD (US); ANGELA MELTON-CELSA, BETHESDA, MD (US)

Correspondence Address: EDWARDS & ANGELL, LLP, P.O. BOX 9169, BOSTON, MA, 02209, US

(21) Appl. No.: 09/215,163

(22) Filed: Dec. 18, 1998

Related U.S. Application Data
(60) Provisional application No. 60/068,635, filed on Dec. 23, 1997.

(51) Int. Cl. A61K 39/395; A61K 39/40; A61K 39/42; A61K 39/00

(52) U.S. Cl. 424/184.1

(57) ABSTRACT

The present invention describes the preparation and use of biologically and immunologically active humanized monoclonal antibodies to Shiga toxin, a toxin associated with HC and the potentially life-threatening sequela HUS transmitted by strains of pathogenic bacteria. The present invention describes how these humanized antibodies may be used in the treatment or prevention of Shiga toxin induced diseases. One aspect of the invention is the humanized monoclonal antibody which binds Shiga toxin where the constant regions are IgG1-kappa and the variable regions are murine in origin. Yet another aspect of the invention is expression vectors and host cells transformed with such vectors which express the humanized monoclonal antibodies of the present invention.

### RELATED APPLICATION

This application claims the benefit of U.S. Provisional Application No. 60/068,635, filed Dec. 23, 1997, which is hereby incorporated by reference.

### GOVERNMENT INTEREST

The invention described herein may be manufactured, licensed, and used for governmental purposes without payment of royalties to us thereon.

### FIELD OF THE INVENTION

The invention relates to a family of multi-unit bacterial proteins that are associated with hemorrhagic colitis and the life-threatening sequela, hemolytic uremic syndrome. These proteins, defined as members of the “Shiga toxin family”, have been utilized in the isolation and identification of murine monoclonal antibodies. The invention further relates to the construction of humanized monoclonal antibodies which incorporate the mouse variable regions. It also relates to antibodies to Shiga toxins or toxoids, both monoclonal and polyclonal, and their use in treating, diagnosing, and preventing of disease and infections caused by pathogenic E. coli. Finally, the invention relates to preparing the humanized monoclonal antibodies to proteins of the Shiga toxin family.

### BACKGROUND OF THE INVENTION

Enterohemorrhagic Escherichia coli (EHEC) are associated with food-borne outbreaks of bloody diarrhea or “hemorrhagic colitis” (HC) and the hemolytic uremic syndrome (HUS). (Spika, J. et al., J. Pediatr., 109: 287-291(1986); Remis, R., Ann. Intern. Med., 101:624-626 (1984); Riley, L. et al., N. Engl. J. Med., 308:681-685 (1983)). EHEC infection can be deadly and poses a significant threat to the young and the elderly, who are the most likely to develop serious complications from EHEC infections. Several outbreaks and sporadic cases of HC and HUS have occurred over the past few years, with the largest U.S. outbreak in 1993. In that outbreak, over 500 cases of HC and HUS were traced to contaminated hamburgers from a Jack-in-the Box fast food restaurant. (Centers for Disease Control and Prevention, Morbid. Mortal. Weekly Rep., 42:258(1993)). In Jul. 1996, a large outbreak of EHEC in Japan resulted in over 10,000 infected individuals and 8 deaths. Many Japanese children required hospitalization.

Primarily, HC and HUS are transmitted by the ingestion of contaminated food, particularly undercooked beef products, such as hamburger. (Doyle et al., J. Appl. Environ. Microbiol. 53:2394 (1987); Samadpour et al., J. Appl. Environ. Microbiol. 60:1038 (1994)). With the prevalence of EHEC in cattle and the subjective nature of differentiating between cooked and undercooked hamburgers, a stop at a fast food restaurant or a family barbecue can result in tragedy.

HC and HUS appear to be mediated by the toxin produced by EHEC and Shigella dysenteriae (for a review, see O'Brien and Holmes, Microbiol. Rev., 51:206-220 (1987)). These bacteria produce a family of closely related cytotoxins that collectively will be called “Shiga toxins” for the purpose of this application. Shiga toxins (alternatively, “verotoxins”) have cytotoxic, and enterotoxic activity (Strockbine, N. et al., Infect. Immun., 53:135-140 (1986)).

Based on the exhibition of immunological non-cross-reactivity, the Shiga toxins have been divided into two groups. (Strockbine et al., supra). These groups have been designated Shiga toxin type 1 (Stx1) and Shiga toxin type 2 (Stx2). The Stx1 group contains the prototype Stx1 toxin from EHEC as well as the Shiga toxin from Shigella dysenteriae type 1. In recent years, other types of toxins have been discovered and considered to be members of the Stx2 group. These are Stx2e, Stx2c, and Stx2d. (Lindgren et al., Infection and Immunology, 61:3832 (1993); Schmitt, C. et al., Infect. Immun., 59:1065-1073 (1991); Marques, L. et al., FEMS Lett., 44:33-38 (1987)).

For the purposes of this application the term “Shiga toxin” encompasses Shiga toxin and any other toxins in the Stx1 or Stx2 group or their variants. The abbreviation “Stx” will refer to the toxin protein itself.

Despite this knowledge about the results of exposure to these toxins, currently there is no known cure or vaccine for HC or HUS. Antibiotics may even make the severe complications worse by increasing toxin release from bacteria. Thus, there is a need for a compound to prevent or to treat the complications of EHEC produced by Shiga toxin. Such a compound could be used to treat infected patients and decrease the systemic effects of toxin on the CNS, blood and kidneys. In addition, if the toxin could be neutralized, antibiotics could be safely given to kill the bacteria in the GI tract. Such a compound could also be used to prevent infectious complications, by treating exposed or high risk individuals before they acquire EHEC infection. Such individuals would include children in day care or the elderly in nursing homes, where a case of EHEC diarrhea has been detected. These individuals are at increased risk to develop severe complications and spread of EHEC in these environments is not unusual.

The knowledge of the immunological cross reactivity of the Shiga toxins offers a tantalizing prospect for pharmacological approaches to EHEC treatment. Currently, there are no known prophylactic or therapeutic agents available for this disease. Accordingly, there is a need in the art to provide monoclonal antibodies that can bind to Shiga toxins which could prevent or lessen the devastating effects of these toxins. There is also a need in the art for data concerning the binding site of such antibodies so that other antibodies with similar abilities can be identified and isolated.

There is a related need in the art for humanized or other chimeric human/mouse monoclonal antibodies. In well publicized studies, patients administered murine anti-TNF (tumor necrosis factor) monoclonal antibodies developed anti-murine antibody responses to the administered antibody. (See, e.g., Exley A. R., et al., Lancet 335:1275-1277 (1990)). This type of immune response to the treatment regimen, commonly referred to as the HAMA response (for human anti-mouse antibodies), decreases the effectiveness of the treatment and may even render the treatment completely ineffective. Humanized or chimeric human/mouse monoclonal antibodies have been shown to significantly decrease the HAMA response and to increase the therapeutic effectiveness. (LoBuglio et al., P.N.A.S. 86:4220-4224 (Jun. 1989)).

### SUMMARY OF THE INVENTION

To satisfy these needs in the art, applicants provide humanized mouse monoclonal antibodies to Shiga toxin 1 and Shiga toxin 2. In addition, this invention sets forth the variable region associated with Shiga toxin neutralization as well as the specific complementarity determining regions (CDRs) of the variable region. The present invention particularly provides human monoclonal antibodies, derived from the mouse monoclonal antibodies 13C4 and 11E10, which are specifically reactive with Stx1 and Stx2, respectively.

In another aspect, the invention includes a pharmaceutical composition and a method of treatment with such composition which will prevent or treat Shiga toxin induced disease.

It is to be understood that both the foregoing general description and the following detailed description are exemplary and explanatory and are intended to provide further explanation of the invention as claimed.

The accompanying drawings are included to provide a further understanding of the invention and are incorporated in and constitute a part of this specification. They illustrate several embodiments of the invention and, together with the description, serve to explain the principles of the invention.

### BRIEF DESCRIPTION OF THE DRAWINGS

FIG. 1 provides a schematic representation of the general cloning strategy used for cloning the variable region gene fragments of both the 11E10 and 13C4 antibodies.

FIG. 2 lists the oligonucleotide sequences used for PCR amplification of the variable regions of the 13C4 antibody.

FIG. 3 recites the DNA and amino acid consensus sequences for the variable regions of both the heavy and light chains of the 13C4 antibody. The DNA consensus sequence was determined using automated fluorescent dye-terminator sequence reactions of the amplified cDNAs analyzed with an ABI 373 sequencing instrument. The CDRs were identified according to the nomenclature of Kabat and Wu (Kabat, et al. (1991), Sequences of Proteins of Immunological Interest, Vol. I, 5th edition, U.S. Department of Health and Human Services) and are located as follows:

| | |
|---|---|
| Heavy Chain CDRs: | CDR1-aa31-35 |
| (SEQ ID NO: 19) | CDR2-aa50-66 |
| | CDR3-aa99-111 |
| Light Chain CDRs: | CDR1-aa24-34 |
| (SEQ ID NO: 21) | CDR2-aa50-56 |
| | CDR3-aa89-97 |

FIG. 4 illustrates the strategy for construction of the mammalian expression vector tKMC249A containing the variable regions of the 13C4 antibody.

FIG. 5 lists the oligonucleotides used for PCR amplification of the variable regions of the 11E10 antibody.

FIG. 6 recites the DNA and amino acid consensus sequences for the variable regions of both the heavy and light chains of the 11E10 antibody. The DNA consensus sequence was determined using automated fluorescent dye-terminator sequence reactions of the amplified cDNAs analyzed with an ABI 373 sequencing instrument. The CDRs were identified according to the nomenclature of Kabat and Wu (Kabat and Wu; supra) and are located as follows:

| | |
|---|---|
| Heavy Chain CDRs: | CDR1-aa31-35 |
| (SEQ ID NO: 44) | CDR2-aa50-66 |
| | CDR3-aa99-108 |
| Light Chain CDRs: | CDR1-aa24-40 |
| (SEQ ID NO: 42) | CDR2-aa56-62 |
| | CDR3-aa95-103 |

FIG. 7 illustrates the strategy for construction of the mammalian expression vector pACE4 containing the variable regions of the 11E10 antibody.

FIG. 8 shows the anti-Stx1 13C4 antibody production ELISA and demonstrates that the H13C4 cell line produces an antibody consisting of human IgG and kappa constant domains.

FIG. 9 shows the anti-Stx1 13C4 toxin binding activity ELISA and demonstrates that the antibody produced by the H13C4 cell line binds to Stx1 toxin.

FIG. 10 shows the anti-Stx2 11E10 antibody production ELISA and demonstrates that the H11E10 cell line produces an antibody consisting of human IgG and kappa constant domains.

FIG. 11 shows the anti-Stx2 11E10 toxin binding activity ELISA and demonstrates that the antibody produced by the H11E10 cell line binds to Stx2 toxin.

FIG. 12 illustrates the strategy for construction of the plasmid pCDNA3mut.LCPL.LCVK.

FIG. 13 illustrates the strategy for construction of the plasmid pCDNA3mut.HCPL.HCV2b.

FIG. 14 illustrates the strategy for construction of the plasmids pSUN9 and pSUN9:kappa.

FIG. 15 illustrates the strategy for construction of the plasmids pSUN10 and pSUN10:IgG1.

FIG. 16 illustrates the strategy for construction of the plasmid pJRS311.

FIG. 17 illustrates the strategy for construction of the plasmid pJRS315.

Additional features and advantages of the invention will be set forth in the description which follows. The objectives and other advantages of the invention will be realized and attained by the composition of matter and process particularly pointed out in the written description and the claims hereof as well as the appended drawings. To achieve these and other advantages and in accordance with the purpose of the invention, as embodied and broadly described, the invention describes monoclonal humanized antibodies which bind Shiga toxin proteins.

### DETAILED DESCRIPTION OF THE INVENTION

The present invention relates to humanized monoclonal antibodies that bind to Shiga toxin proteins, and the use of such antibodies in the treatment or prevention of Shiga toxin-induced diseases. Antibodies are proteinaceous structures made up of two heavy and two light chains. The five different classes of higher vertebrate antibodies—IgM, IgD, IgG, IgA and IgE—are distinguished by their heavy chains (mu, delta, gamma, alpha and epsilon, respectively). Each class has further subclasses; IgG, for example, may be IgG1, IgG2, IgG3, or IgG4, where the heavy chain is gamma 1, gamma 2, gamma 3 or gamma 4, respectively. The pair of light chains in these classes or subclasses may be either kappa or lambda.

Antibodies are further divided into a constant region and a variable region. For both the heavy and light chains, the carboxy-terminal ends make up the constant sequence region, while the amino terminal ends contain the variable sequence region. Within these variable regions, the complementarity determining regions (CDRs) are located which are primarily responsible for the observed antigen binding which is characteristic of antibodies.

“Humanized” monoclonal antibodies mean monoclonal antibodies originally from a non-human source to which human components have been substituted. In a preferred embodiment, the humanized monoclonal antibodies of this invention comprise variable regions which derive from non-human sources and constant regions which derive from human sources.

As set forth above, Shiga toxin proteins (Stx) refer to the family of multi-unit bacterial proteins produced by EHEC and Shigella dysenteriae which are associated with outbreaks of Shiga toxin induced diseases. Shiga toxin proteins are meant to encompass Stx of Shigella dysenteriae-type 1 and Stx type 1 and type 2, and type 2 variant toxins of E. coli.

Shiga toxin-induced diseases of humans include bloody diarrhea, hemorrhagic colitis, hemolytic uremic syndrome, and thrombotic thrombocytopenic purpura.

In a more specific embodiment, the invention relates to humanized monoclonal antibodies that have the same binding specificity of at least two well characterized murine monoclonal antibodies. These two monoclonal antibodies were developed in Dr. Alison O'Brien's laboratory as set forth in Strockbine, N. A. et al., Infection and Immunity, 1985, 50:695-700 and Perera, L. P. et al., J. Clinical Microbiol., 1988, 26:2127-2131, and have been deposited at the ATCC as set forth below.

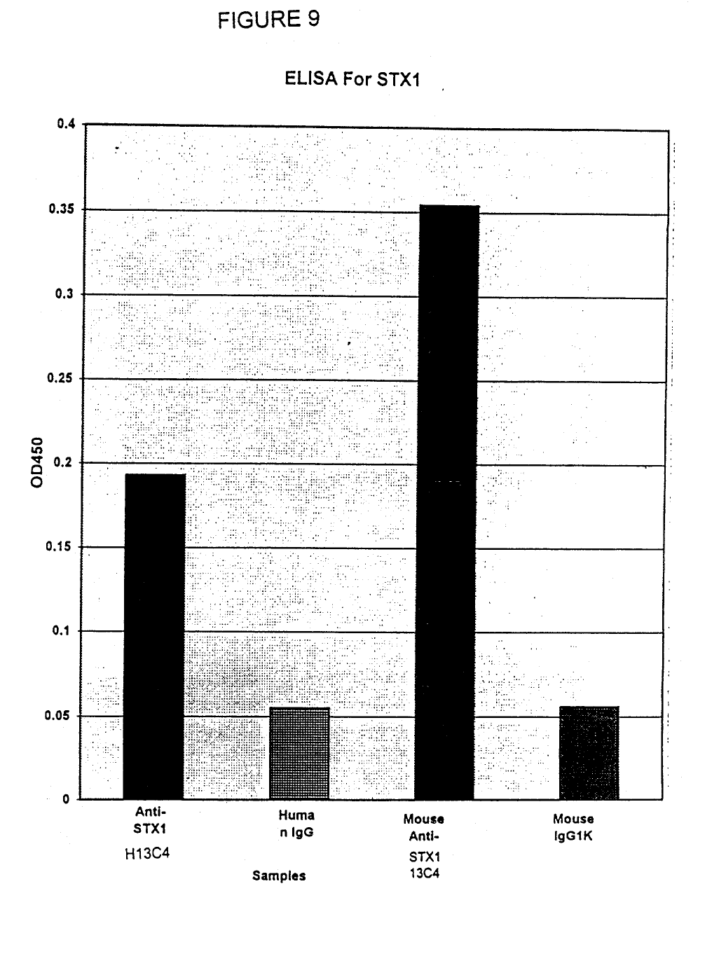

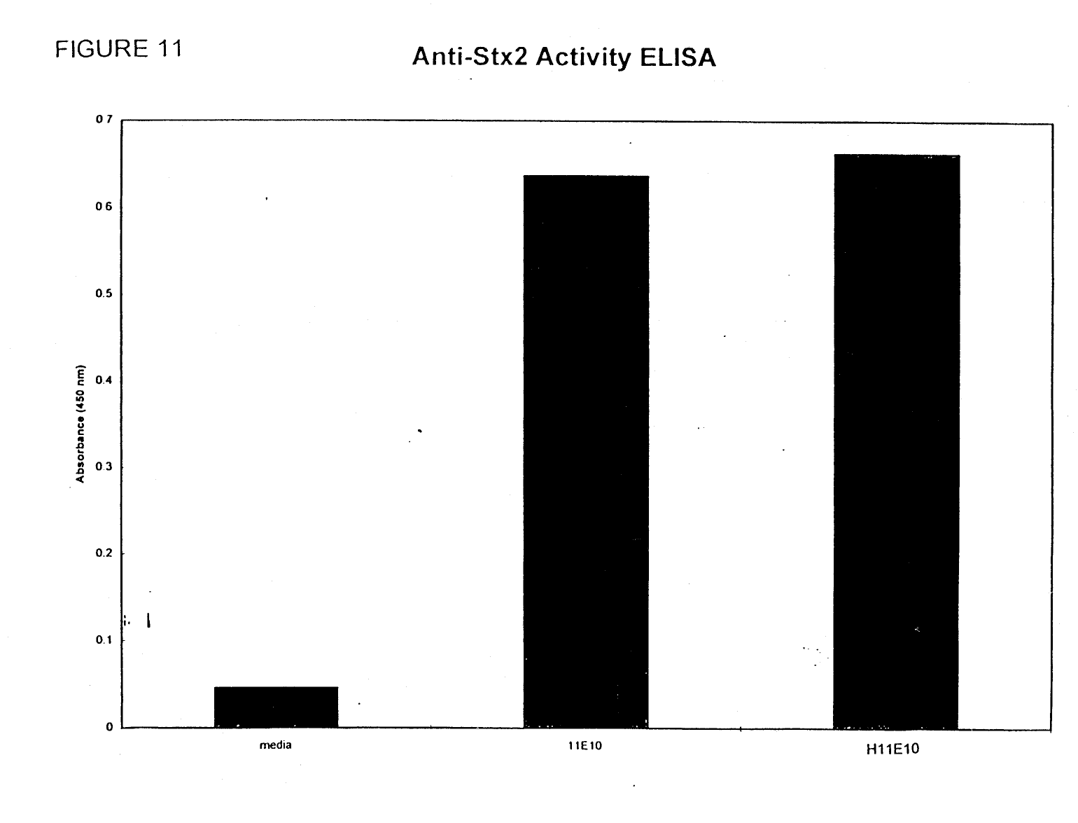

By “same binding specificity” is meant a level of binding sufficiently detectable in a standard binding assay to distinguish between toxin binding and non-specific background binding as exemplified by appropriate controls (for examples, see FIG. 9 and FIG. 11). Those of ordinary skill in the art can readily test for binding levels using routine skills and techniques.

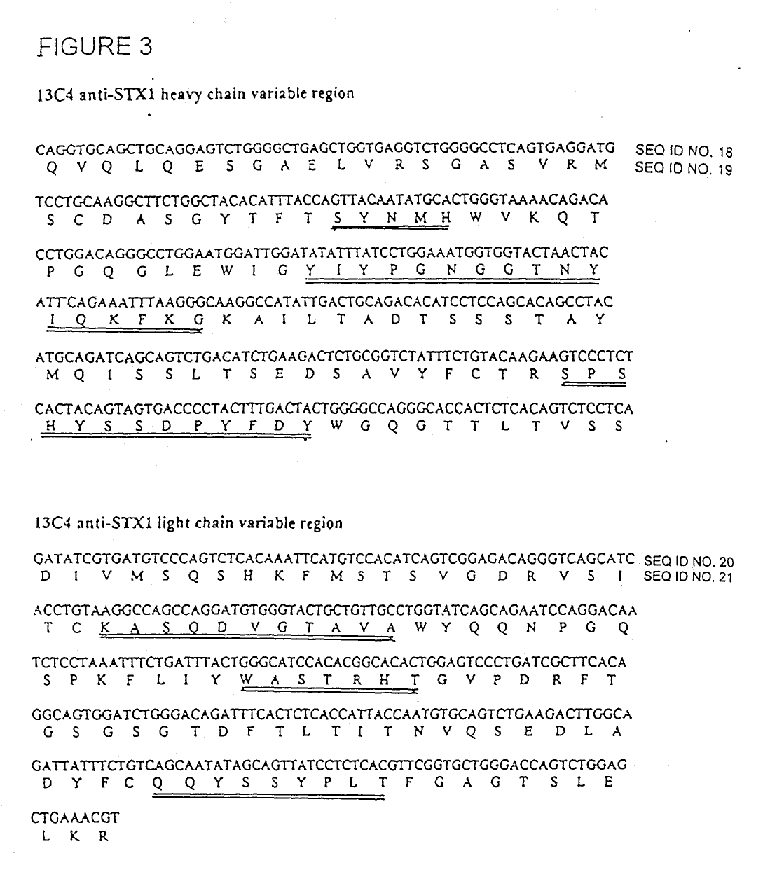

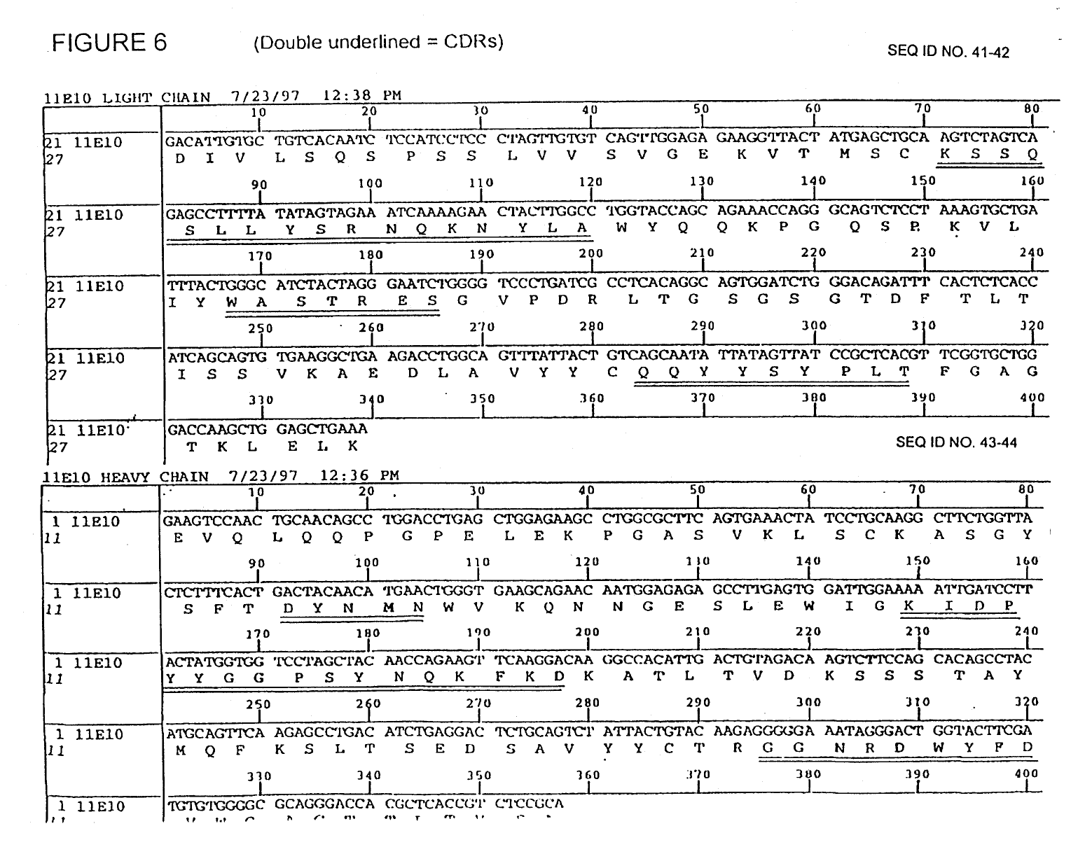

In another preferred embodiment, the humanized antibodies are characterized by their structural features. In one aspect of this embodiment, the constant region is a human constant region and the variable region derives from a rodent, preferably a mouse. Although not limited to these, the four variable regions set forth in the specification, particularly in FIG. 3 and FIG. 6, are preferred. Of course, the invention includes modifications (i.e., deletions, additions, and substitutions) that do not appreciably diminish the characteristic binding associated with the exemplified variable regions.

In yet another embodiment, the CDRs of the variable region are employed in the invention. As noted above, CDRs are located in the variable regions of both the light and heavy chains and are responsible for antigen binding. In view of the ready ability of those in this art to determine the CDR regions, the present invention is not limited to the CDRs specifically set forth. Indeed, the vectors created by the applicants and described in great detail below are sufficient for use with any CDRs of non-human antibodies to Shiga toxins.

In the most preferred embodiment of the present invention, the humanized monoclonal antibody derives its constant regions from IgG1-kappa and its variable regions from all or part of the sequences as set forth in FIGS. 3 and 6. In further embodiments, the invention also relates to the expression vectors which code for these monoclonal humanized antibodies, and to host cells which have been transformed with such expression vectors.

Finally, the invention comprises the pharmaceutical compositions and the methods of use of these humanized monoclonal antibodies to treat or prevent Shiga toxin-producing bacterial infections. As a pharmaceutical, this invention includes but is not limited to diluents and carriers known in the art, such as saline and sucrose solutions appropriate for application to patients. As used herein, “patients” refers to any susceptible mammal, such as dogs, horses, mice, etc., but is particularly preferred to mean humans.

The invention further involves the administration of a therapeutically effective amount, as well as a prophylatically effective amount, of the humanized monoclonal antibody of the invention. As those in the art would recognize, a therapeutically effective amount is a dose that ameliorates edema, thrombocytopenia, and uremia associated with EHEC-mediated HUS. Similarly, a prophylactically effective amount is a dose that prevents exposed individuals from developing these symptoms.

The following examples illustrate specific embodiments of the invention.

Humanization of the Anti-Stx1 Antibody 13C4

The following examples 1-3 relate to anti-Stx1 antibody 13C4 and its humanized counterpart H13C4.

### EXAMPLE 1

### Cloning of the 13C4 Variable Region cDNAs

The hybridoma cell producing the “13C4” antibody (Anti-Stx1) was deposited on Dec. 2, 1987, at the American Type Culture Collection, Rockville, Md. under Accession No. CRL 1794, and can be obtained from the ATCC, or, as here, from Dr. Alison O'Brien (for details of hybridoma preparation, see Strockbine, N. A. et al., Infection and Immunity, 50:695-700 (1985)). A vial of cells was thawed and resuspended in IMDM (Mediatech) complete media supplemented with 10% FBS (Irvine).

Total RNA was isolated from 1×10⁷ “13C4” cells using the Midi RNA Isolation kit (Qiagen) following the manufacturer's procedure. The RNA was dissolved in 10 mM Tris, 0.1 mM EDTA (pH 8.4) containing 0.03 U/μg Prime RNase Inhibitor (5′-3′) to a final concentration of 0.25 μg/μl.

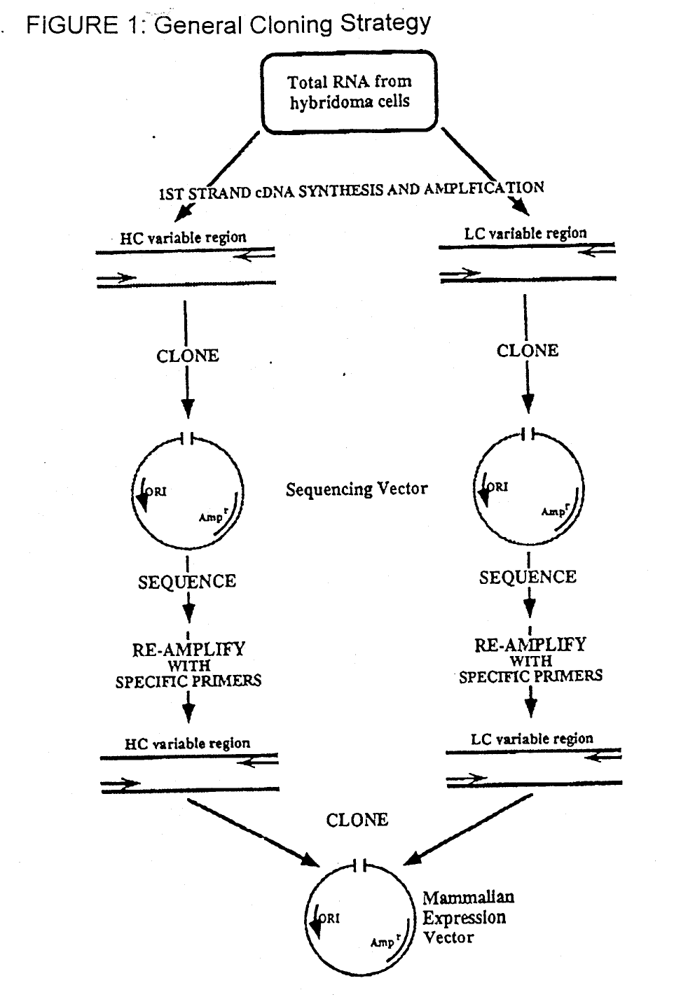

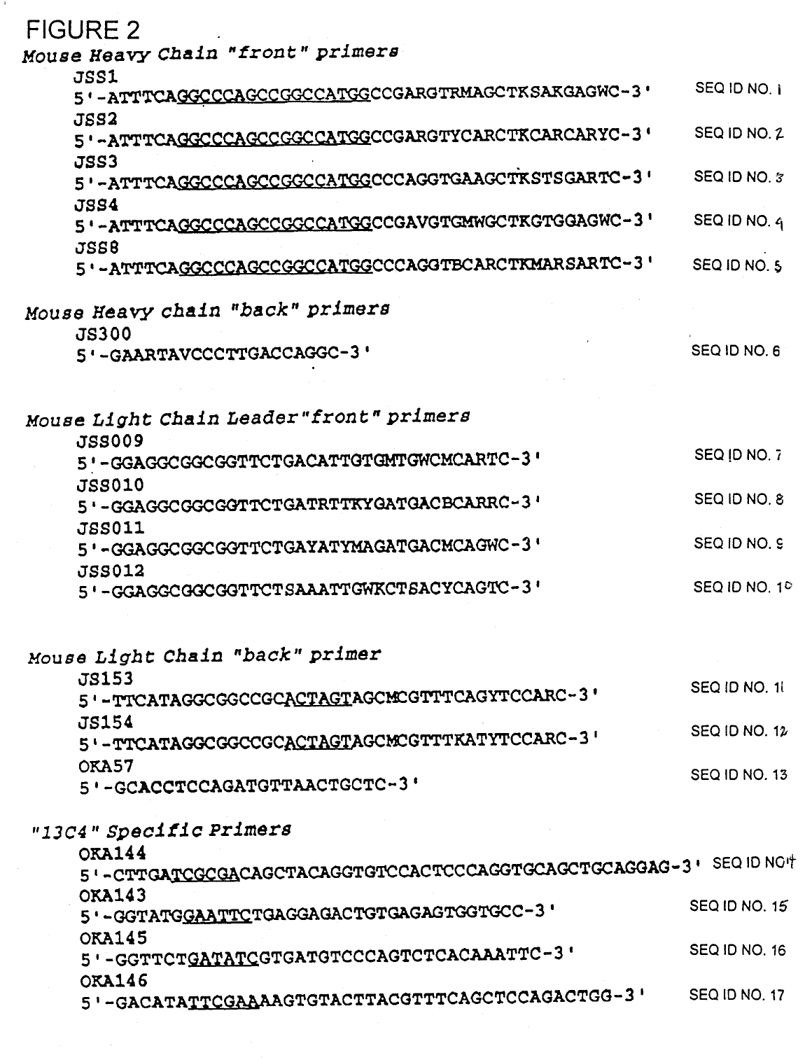

FIG. 1 shows the strategy for cloning the variable region gene fragments and FIG. 2 lists the oligonucleotide primers used. The “13C4” total RNA (2 μg) was converted to cDNA by using Superscript ll-MMLV Reverse Transcriptase (Life Technologies) and mouse kappa (oKA57, SEQ ID NO:57) and mouse CH1 (JS300, SEQ ID NO: 6) specific priming, according to the manufacturer's procedures. The first strand cDNA synthesis products were then purified using a membrane concentrator device (Amicon Centricon 30 or Millipore UltraFree 15). Of the cDNA recovered, 3 μl was used as template DNA for PCR. Typical PCR amplification reactions (100 μl) contained template DNA, 50 pmoles of the appropriate primers (JSS9, JSS10, JSS11, JSS12, JS153 & JSS154—SEQ ID NO:9-12 for light chains; JSS1, JSS2, JSS3, JSS4, JSS8 and oKA-143—SEQ ID NO:1-5 and SEQ ID NO:15 for heavy chains), 2.5 units of ExTaq polymerase (PanVera), 1× ExTaq reaction buffer, 200 μM dNTP, and 2 mM MgCl₂. Each of the templates was denatured by an initial incubation at 96° C. for 1 min. The heavy chain products were amplified by 40 or 50 thermal cycles of 59 to 72° C. for 30 sec., 72° C. for 30 sec., then 96° C. for 1 min. and a final extension step at 72° C. for 5 min. The light chain products were amplified by 6 thermal cycles of 46, 48 or 54° C. for 30 sec., 72° C. for 30 sec., then 96° C. for 1 min followed by 35 step cycles of 60° C. for 1 min, then 96° C. for 1 min. and a final extension step at 72° C. for 5 min. The PCR products from the successful reactions were purified using the Wizard PCR Purification system (Promega) as per the manufacturer's procedure.

The heavy chain and light chain PCR products (approximately 400 bp) were then cloned into a bacterial vector for DNA sequence determination. Ligations of the PCR fragments were carried out into the pCR2.1 T/A style cloning vector (Invitrogen) following the manufacturer's procedures using 1:1, 3:1 and 5:1 insert to vector molar ratios. One half of each of the ligation reactions was used to transform competent XL 1 Blue cells.

Heavy chain plasmid clones containing DNA inserts were identified using diagnostic restriction enzyme digests with EcoRI (New England Biolabs). The DNA sequence of plasmids (tKMC217B) containing inserts of the appropriate size (400 bp) were then determined. The final consensus DNA sequence of the heavy chain variable regions is shown in FIG. 3.

The light chain PCR products were identified differently. The hybridoma cell line that expresses the “13C4” antibody was made by fusing mouse splenocytes with the SP2/0 myeloma cell line. The SP2/0 cell line transcribes a pseudogene for the kappa light chain. The pseudogene transcript, when converted to cDNA by RT-PCR, contains an AflIII restriction site. Light chain candidate clones (tKMC226 & 227) were digested with AflIII (New England Biolabs) using the manufacturer's procedures to identify clones containing inserts of the appropriate size (403 bp; no AflIII site is present in these products). The final consensus DNA sequence of the light chain variable regions is shown in FIG. 3 with the CDRs indicated by underlining.

### EXAMPLE 2

### Construction of the Expression Vector tKMC249A

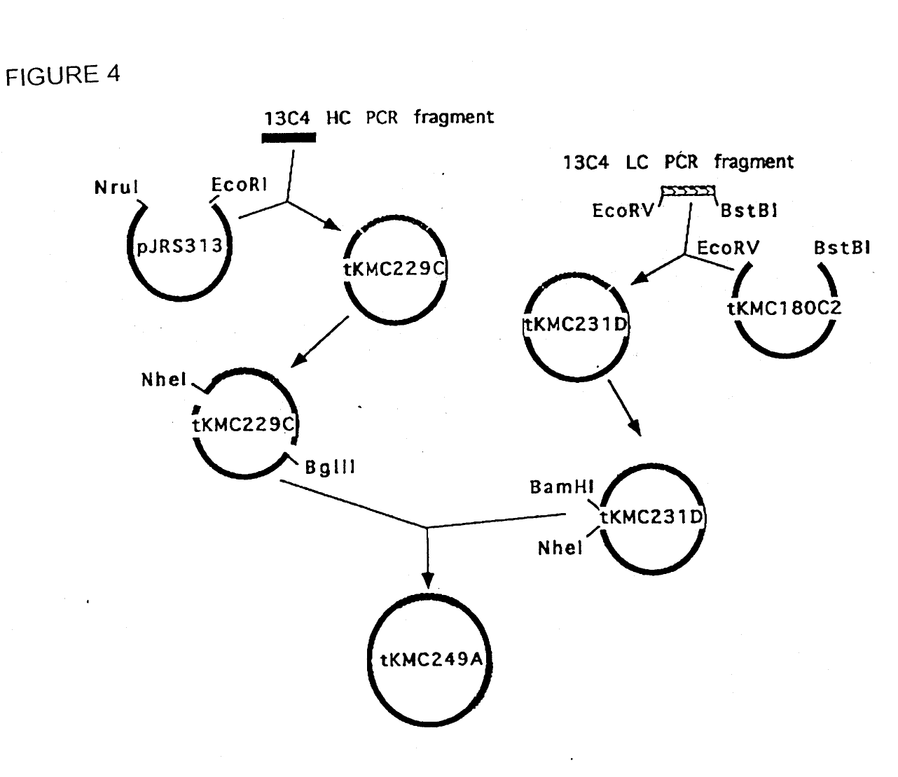

The heavy and light chain variable regions were then subcloned into mammalian expression plasmid vectors for the production of recombinant chimeric mouse/human antibody molecules. The vectors result in the production of recombinant antibody molecules under the control of CMV transcriptional promoters. The heavy chain molecules are direct cDNA constructs that fuse the variable region sequence directly into the human IgG1 constant domain. The light chain molecules, on the other hand, have a mouse kappa intron region 3′ of the variable region coding fragment. After splicing, the variable region becomes fused to a human kappa constant region exon (FIG. 4). The selectable marker for the vector in mammalian cells is aminoglycoside phosphotransferase (neo), using the drug G418 (CellTech).

A. Creation of the Expression Vectors

To create the heavy and light chain expression vectors required DNA fragment ligations and site directed mutagenesis steps. The result was vectors that express both antibody chains with CMV promoter driven transcription. Neomycin resistance serves as a dominant selectable marker for transfection of mammalian cells. In addition, these vectors have been designed to allow convenient cloning of any light chain variable region as EcoRV/BstBI fragment, any heavy chain variable region as a NruI/EcoRI fragment, and any heavy chain constant domain as an EcoRI/NotI fragment. These restriction sites were chosen because they occur rarely (if ever) in human and mouse variable regions. There is a mouse J region/kappa intron fragment fused to a human kappa exon so that after post-transcriptional splicing a mouse human chimeric kappa light chain is produced. Lastly, the vectors were designed to facilitate excision (BglII/NheI) of a whole antibody expression cassette from one vector to be ligated into a second vector cut with BamHI/NheI, creating an expression vector with the apparatus for both chains.

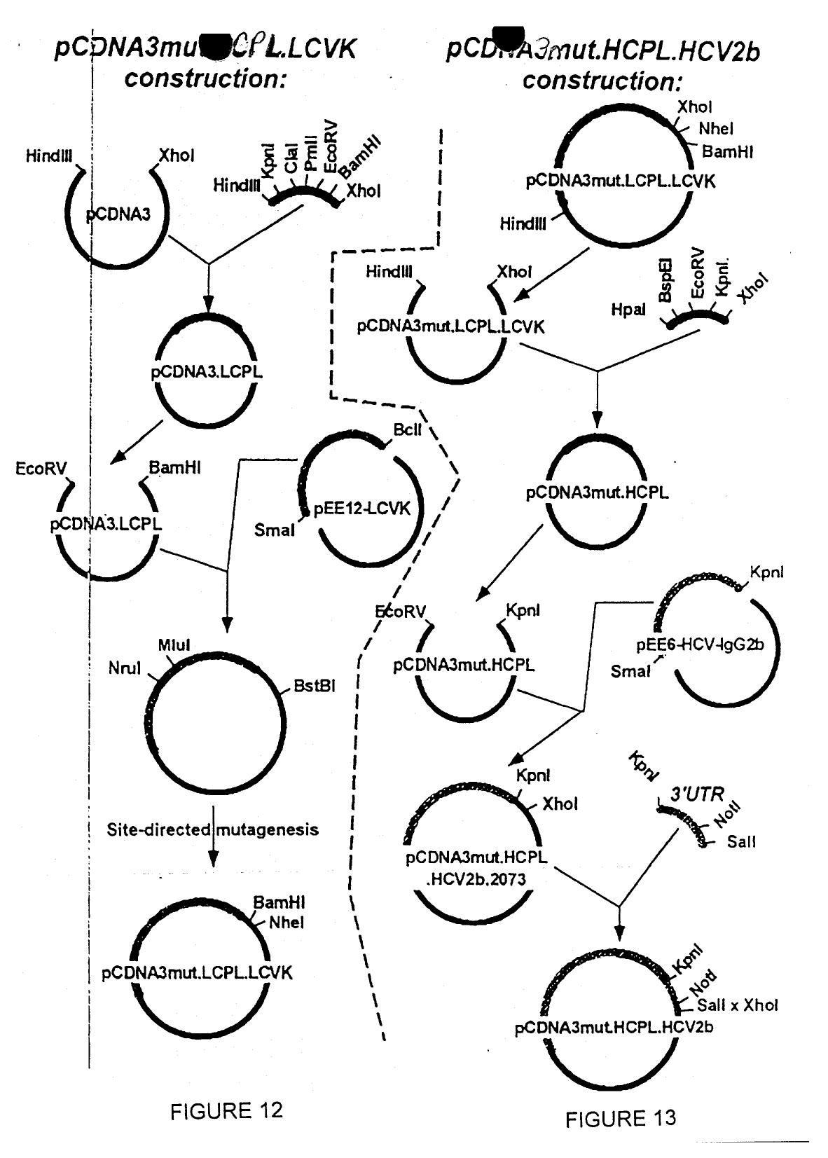

The backbone of the vector was the plasmid pCDNA3 (Invitrogen). This plasmid was cut with HindIII/XhoI and a “light chain polylinker” DNA fragment was inserted to create the starting “light chain vector” pCDNA3.LCPL (see FIG. 12). This linker contained the restriction sites HindIII, KpnI, ClaI, PmlI, EcoRV, XmaI, BamHI and XhoI to facilitate subsequent cloning steps. A SmaII/BclI DNA fragment containing a light chain leader, mouse anti-CKMB kappa light chain genomic fragment, and 3′ UTR was cloned into the EcoRV/BamHI sites of pCDNA3.LCPL. The mouse kappa intron, exon, and 3′ UTR in this fragment was derived from LCPXK2 received from Dr. Richard Near (Near, R. I. et al., 1990, Mol. Immunol. 27:901-909). Mutagenesis was then performed to eliminate an NruI (209), MluI (229), and BstBI (2962) and to introduce an NheI (1229) and a BamHI (1214) site to create the plasmid pCDNA3mut.LCPL.LCVK (see FIG. 12).

A second “heavy chain vector” pCDNA3mut.HCPL was constructed from the pCDNA3mut.LCPL.LCVK plasmid by replacing the light chain expression region (HindIII/XhoI) with a “heavy chain polylinker” consisting of restriction sites HpaI, BspEI, EcoRV, KpnI, and Xhol. This plasmid was digested with EcorRV and KpnI. A SmaI/KpnI digested DNA fragment containing a heavy chain leader and an anti-CKMB IgG2b mouse heavy chain genomic fragment was then ligated into the EcoRV/KpnI digested plasmid. A KpnI/SalI oligonucleotide fragment containing a 3′ UTR and a NotI upstream of the SalI site was subsequently cloned into the KpnI/XhoI digested plasmid, (knocking out the XhoI site), to create the plasmid pCDNA3mut.HCPL.HCV2b (see FIG. 13).

A human kappa light chain constant domain was then cloned into pCDNA3mut.LCPL.LCVK as a EcoNI/XhoI fragment generating the plasmid tKMC180C2. A human IgG1 constant domain was cloned into pSUN10 as a EcoRI/NotI fragment creating the plasmid pJRS313. The variable regions of 13C4 were cloned into these two vectors (as described above). A BglII/NheI fragment from the human heavy chain vector tKMC229C was then cloned into the human light chain vector tKMC231D cut BamHI/NheI to create tKMC249A (see FIG. 4).

B. Subcloning the Variable Regions Into the Expression Vectors

The variable region gene fragments were re-amplified by PCR using primers that adapted the fragments for cloning into the expression vectors (see FIGS. 2 and 4). The heavy chain front primer (oKA143, SEQ ID NO:15) includes a 5′ tail that encodes the C-terminus of the heavy chain leader and an NruI restriction site for cloning, while the heavy chain reverse primer (oKA144, SEQ ID NO:14) adds a 3′ EcoRI restriction site for cloning. The light chain front primer (oKA145, SEQ ID NO:16) introduces an EcoRV restriction site at the N-terminus of the light chain variable region for cloning, while the light chain reverse primer (oKA146, SEQ ID NO:17) adds a 3′ DNA sequence for the joining region-kappa exon splice junction followed by a BstBI restriction site for cloning.

PCR reactions were performed with reagents as described above and with templates of 1-2 ng of PvuI (New England Biolabs) digested plasmid; each of these templates was denatured by an initial one minute incubation at 96° C. The heavy chain products were amplified by 35 thermal cycles of 55 or 60° C. for 30 sec., 72° C. for 30 sec., then 96° C. for 1 min and a final extension step at 72° C. for 5 min. The light chain products were amplified by 8 thermal cycles of 55 or 60° C. for 30 sec., 72° C. for 30 sec., then 96° C. for 1 min followed by 30 step cycles of 60° C. for 1 min, then 96° C. for 1 min, and a final extension step at 72° C. for 5 min.

The 13C4 heavy chain PCR product (approximately 400 bp) was purified using Qiaquick PCR Purification columns (Qiagen) as described by the manufacturer's instructions and subsequently digested with NruI and EcoRI (New England Biolabs). The digested PCR products were purified using the Wizard PCR Purification system (Promega) as per manufacturer's procedure and ligated into NruI/EcoRI digested and gel-purified pJRS313, resulting in plasmid tKMC229C (see FIG. 4). The final consensus DNA sequence of the heavy chain variable region and proper splicing of the restriction sites were confirmed in this construct.

The 13C4 light chain PCR product (approximately 350 bp) was purified using Qiaquick PCR Purification columns (Qiagen) as described by the manufacturer's instructions and subsequently digested with EcoRV and BstBI (New England Biolabs). The digested PCR products were purified using Qiaquick PCR Purification columns (Qiagen) as per manufacturer's procedure and ligated into EcoRV/BstBI digested and gel-purified tKMC180C2 (as described above), resulting in plasmid tKMC231 D (see FIG. 4). The final consensus DNA sequence of the light chain variable region and proper splicing of the restriction sites were confirmed in this construct.

### EXAMPLE 3

### Stable Production of Recombinant Chimeric Mouse/Human 13C4 Antibody

A. Transfection of NSO Cells

The plasmid tKMC249A was transfected into NSO cells (Baxter International, Inc., Durante, Calif.) using electroporation after linearization with PvuI (New England Biolabs). 40 micrograms of the digested plasmid was mixed with 1×10⁷ cells in a total volume of 800 microliters in a 0.4 centimeter cuvette and subjected to a pulse of 250 mA, 960 μF. The cells were plated out after 24 hours into 96-well tissue culture plates, 6 plates with 200 μl/well, and incubated at 37° C. and 10% CO₂. As colonies appeared, the supernatants were assayed for the production of “humanized” antibody and for the capability of the expressed antibody to bind to Stx1.

B. Assay for Antibody Production

Antibody production and activity assays for the stable transfectants were performed as described below. These assays demonstrate that the transfection of cells with this plasmid construct can result in the production of a stable cell line that produces a humanized chimeric version of the 13C4 mouse hybridoma antibody (designated H13C4).

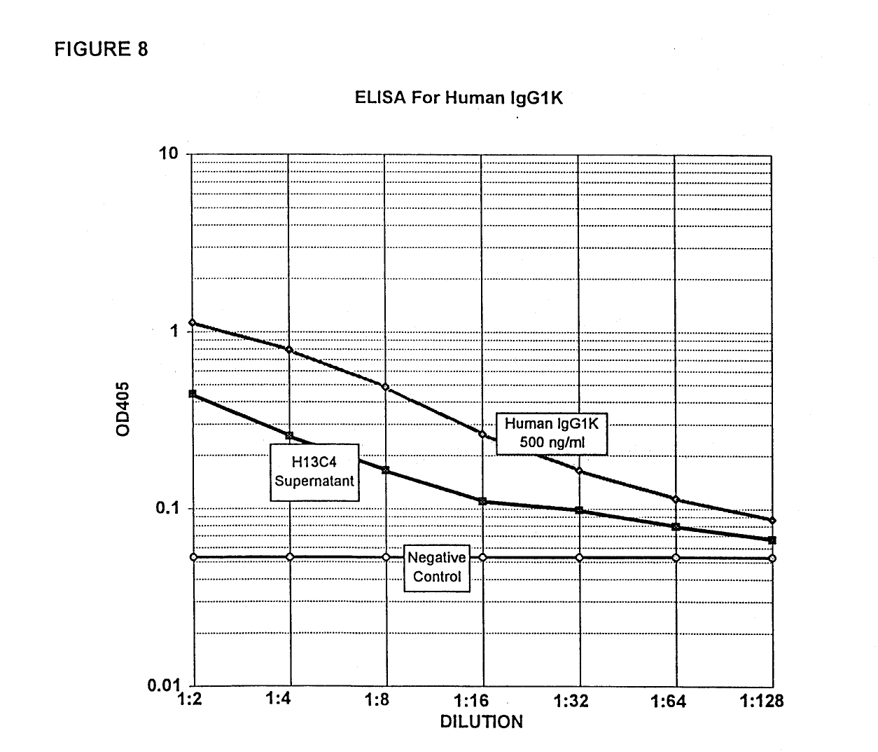

Antibody production ELISA assays were performed in 8-well strips from 96-well microtiter plates (Maxisorp F8; Nunc, Inc.) coated at a 1:500 dilution with Goat anti-Human IgG antibody (Pierce or Biodesign International) using a Tris-HCl coating buffer, pH 8.5. The plates were covered and incubated overnight at 4° C. Plates were then washed once with a wash storage buffer (Tris-HCl/NaCl/0.1% NaN₃). 50 microliters of culture supernatant was then applied to each well that had been filled previously with 50 microliters of a sample/conjugate diluent (Tris-HCl/NaCl/gelatin/Tween-20). The plates were allowed to incubate for 30 to 60 minutes on a rotator at room temperature. They were then washed three times with a wash solution (imidazole/NaCl/Tween-20). A goat anti-human kappa-HRP (Southern Biotechnologies) conjugate was diluted 1:250 in the sample/conjugate diluent and 100 microliters was added to the wells. The plates were incubated on a rotator for 30 to 60 minutes at room temperature. They were washed 6 times using the wash buffer, and then incubated with 100 μL/well of ABTS developing substrate (Kirkgaard & Perry Laboratories) for 8 minutes at room temperature. The reaction was stopped with 100 μL/well of diluted Quench buffer (Kirkgaard & Perry Laboratories). The absorbance value at 405 nm was determined using an automated microtiter plate ELISA reader (Ceres UV900HI, Bioteck, Winooski, Vt.). The controls for the ELISA assay were a human IgG1K myeloma protein (Biodesign International) and supernatant collected from non-transfected NSO cells. This assay (see FIG. 8) demonstrates that the transfection of cells with this plasmid construct results in the cells producing a molecule containing both human heavy chain (IgG) and light chain (kappa) domains.

The supernatants were then assayed for the ability of the expressed antibodies to bind to Stx1 protein by ELISA. The activity assays were preformed in 8-well strips from 96-well microtiter plates (Maxisorp F8; Nunc, Inc.) coated with approximately 0.1 μg/well purified Stx1 received from Alison O'Brien (or obtained as described in Example 7). The plate coating and ELISA procedure was performed in the same manner as the antibody assay above with the substitution of TMB (Kirkgaard & Perry Laboratories) for ABTS as a developing substrate. The absorbance value at 450 nm was determined using an automated microtiter plate ELISA reader (Ceres UV900HI, Bioteck, Winooski, Vt.). As a positive control, the original mouse monoclonal antibody 13C4 was used, and assayed with a goat anti-mouse IgG conjugate (Jackson Laboratories) at a 1:2000 dilution. This assay (see FIG. 9) demonstrates that the transfection of cells with this plasmid construct results in cells producing immunoglobulin that binds to Stx1. Neither mouse nor human IgG1K lacking the anti-Stx variable region bound the toxin.

Humanization of the EHEC Anti-Stx2 Antibody: 11E10

The following examples 4-7 relate to anti-Stx2 and -Stx2 variant antibody 11E10 and its humanized counterpart H11E10.

### EXAMPLE 4

### Cloning of the 11E10 Variable Region cDNAs

The hybridoma cell line producing the “11E10” antibody (Anti-Stx2) was deposited on Aug. 1, 1990, at the American Type Culture Collection, Rockville, Md. under Accession No. CRL 1987, and can be obtained from the ATCC, or, as here, from Dr. Alison O'Brien (for details of hybridoma preparation, see Perera, L. P. et al., J. Clinical Microbiol., 26:2127-2131 (1988)). A vial of the cell line was thawed, washed with serum free medium and then resuspended in IMDM (Mediatech) complete media supplemented with 10% FBS (Irvine).

Total RNA was isolated from 1×10⁷ “11E10” cells using the RNeasy RNA Isolation kit (Qiagen) following the manufacturer's procedure. The RNA was dissolved in 10 mM Tris, 0.1 mM EDTA (pH 8.4) containing 0.03 U/μg Prime RNase Inhibitor (5′-3′) to a final concentration of 0.63 μg/μl.

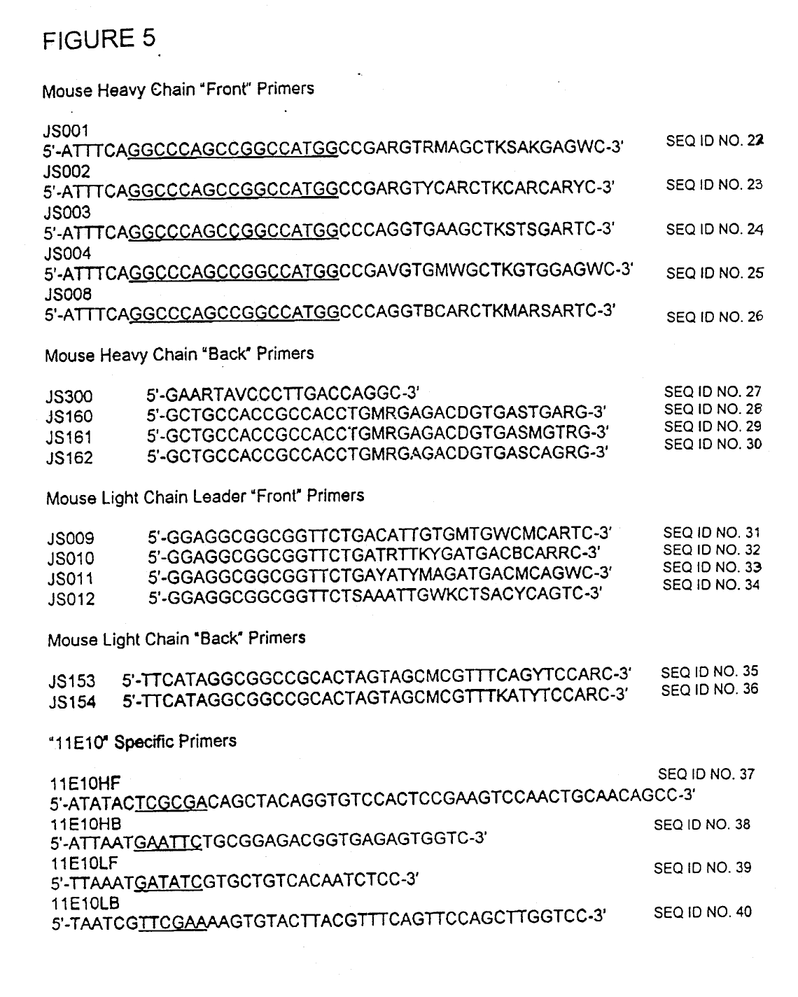

FIG. 1 shows the strategy for cloning the variable region gene fragments and FIG. 5 lists the oligonucleotide primers used. The “11E10” total RNA (2.5 μg) was converted to cDNA by using Superscript ll-MMLV Reverse Transcriptase (Life Technologies) according to manufacturer's procedures. The mouse light chain (JS153, JS154, SEQ ID NO:11 and 12) and mouse heavy chain (JS300, SEQ ID NO:6) were used as specific primers. The first strand cDNA synthesis products were then purified using a Centricon-30 concentrator device (Amicon). Of the 70 μl of cDNA recovered, 3.5 μl was used as template DNA for PCR. Typical PCR amplification reactions (100 μl) contained template DNA, 50 pmoles of the appropriate primers (JS153, JS154 and JS009, JS010, JS011, JS012, SEQ ID NO:7-12 for light chains, JS160, JS161, JS162 and JS001, JS002, JS003, JS004, JS008, SEQ ID NO:28-30, SEQ ID NO:2-5 for heavy chains), 2.5 units of ExTaq polymerase (PanVera), 1× ExTaq reaction buffer, 200 μM dNTP, and 2 mM MgCl₂. The template was denatured by an initial five minute incubation at 96° C. The products were amplified by 35 thermal cycles of 96° C. for 1 min., 55° C. for 30 sec., and 72° C. for 30 sec, followed by 5 min. at 72° C. The PCR products from the successful reactions were purified using the Wizard PCR Purification system (Promega) as per manufacturer's procedure.

The heavy chain PCR products (approximately 400 bp) and the light chain PCR products (approximately 350 bp) were then cloned into a bacterial vector for DNA sequence determination. Ligations of the PCR fragments were carried out into the pCR2.1 T/A style cloning vector (Invitrogen) following the manufacturer's procedures using a 3:1 insert to vector molar ratio. Two μl of the ligation reactions were used to transform the INVαF′ competent cells (Invitrogen) as per the manufacturer's procedure. Plasmid clones containing DNA inserts were identified using diagnostic restriction enzyme digests, with EcoRI (New England Biolabs). The DNA sequence of plasmids containing the heavy chain inserts of the appropriate size (400 bp) was then determined. The final consensus DNA sequence of the heavy chain variable region of 11E10 is shown in FIG. 6 with the CDRs indicated by underlining.

The light chain plasmid clones needed to be further characterized because the hybridoma cell line that expresses the “11E10” antibody was made by fusing mouse splenocytes with SP20 myeloma cells. The SP20 cell line transcribes a pseudogene for the kappa light chain. The pseudogene transcript, when converted to cDNA by RT-PCR, contains an AflIII restriction site. For this reason, the plasmid clones for the light chain variable region were digested with AflIII and those products that did not cut were then submitted for DNA sequencing. The final consensus sequence of the light chain variable region is shown in FIG. 6, with the CDRs indicated by underlining.

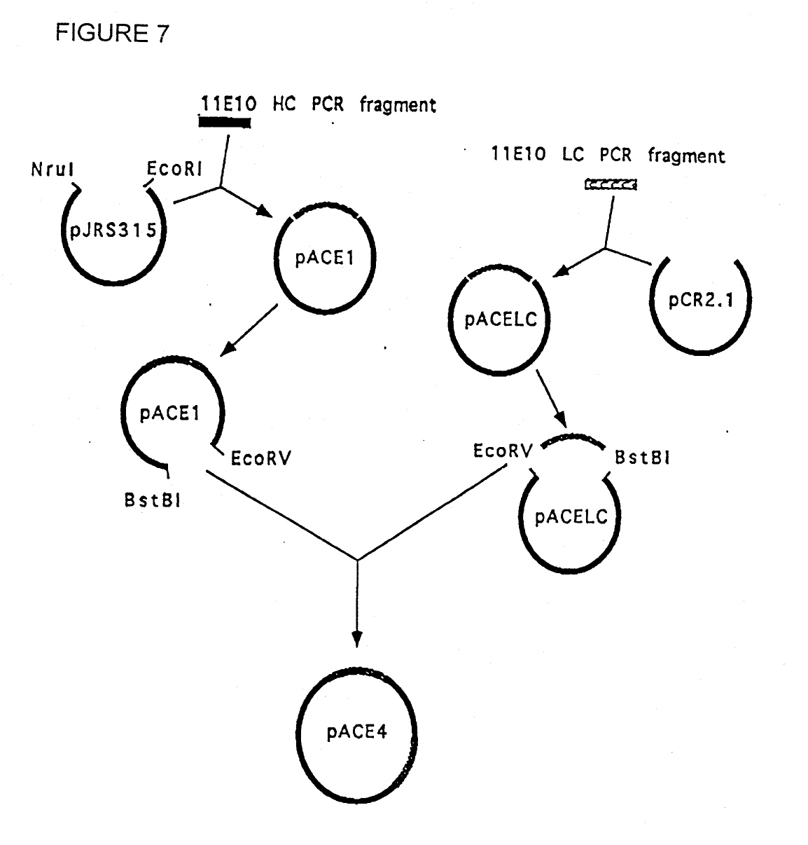

The variable region gene fragments were re-amplified by PCR using primers that adapted the fragments for cloning into the expression vector (see FIGS. 5 and 7). The heavy chain front primer (11E10HF, SEQ ID NO:37) includes a 5′ tail that encodes the C-terminus of the heavy chain leader and an NruI restriction site for cloning, while the heavy chain reverse primer (11E10HB, SEQ ID NO:38) adds a 3′ EcoRI restriction site for cloning. The light chain front primer (11E10LF, SEQ ID NO:39) includes a 5′ tail that encodes the C-terminus of the light chain leader and an EcoRV restriction site at the N-terminus of the light chain variable region for cloning, while the light chain reverse primer (11E10LB, SEQ ID NO:40) adds a 3′ DNA sequence for the joining region-kappa exon splice junction followed by a BstBI restriction site for cloning. PCRs were performed as described above except, following a 5 min. incubation at 96° C., the PCR parameters were 30 thermal cycles of 96° C. for 1 min., 62° C. for 30 sec., and 70° C. for 30 sec., followed by 5 min. at 72° C.

The heavy chain variable region PCR product was then subcloned into a mammalian expression plasmid vector (pJRS315, produced as set forth below in Example 5) for production of recombinant chimeric mouse/human antibody molecules. The resulting vector clone was designated pACE1. Before the light chain variable region could be subcloned into the mammalian expression vector, it was subcloned into the pCR2.1 T/A style cloning vector previously described. The resulting plasmid was designated pACELC and digested, with BstBI/EcoRV, to cut out the light chain variable region. The variable region was then subcloned into the mammalian expression vector containing the “11E 10” heavy chain variable region (pACE1). The final expression vector clone was designated pACE4. This vector results in the production of recombinant antibody molecules under the control of the CMV transcriptional promoters. The heavy chain molecules are direct cDNA constructs that fuse the variable region sequence directly into the human IgG1 constant domain. The light chain molecules, on the other hand, have a mouse kappa intron region 3′ of the variable region coding fragment. After splicing, the variable region becomes fused to a human kappa constant region exon (see FIG. 7). The selectable marker for the vector in mammalian cells is neomycin (G418).

For the production of pACE1, the “11E10” heavy chain PCR product (approximately 400 bp) was digested with NruI and EcoRI (New England Biolabs), purified using a Qiaquick PCR Purification column (Qiagen), as described by the manufacturer, and ligated into NruI/EcoRI digested and gel-purified pJRS315, resulting in plasmid pACE1 (see FIG. 7).

The “11E10” light chain PCR product (approximately 350 bp) was cloned into the T/A cloning vector as per manufacturer's instructions. The resulting clone, pACELC, was digested with EcoRV and BstBI (New England Biolabs) and the light chain fragment was gel-purified. This fragment was then ligated into the EcoRV/BstBI digested and gel-purified pACE1, resulting in plasmid pACE4 (see FIG. 7). The sequence of the heavy and light chain variable regions was verified prior to mammalian cell transfection.

### EXAMPLE 5

### Construction of the Expression Vector pJRS315

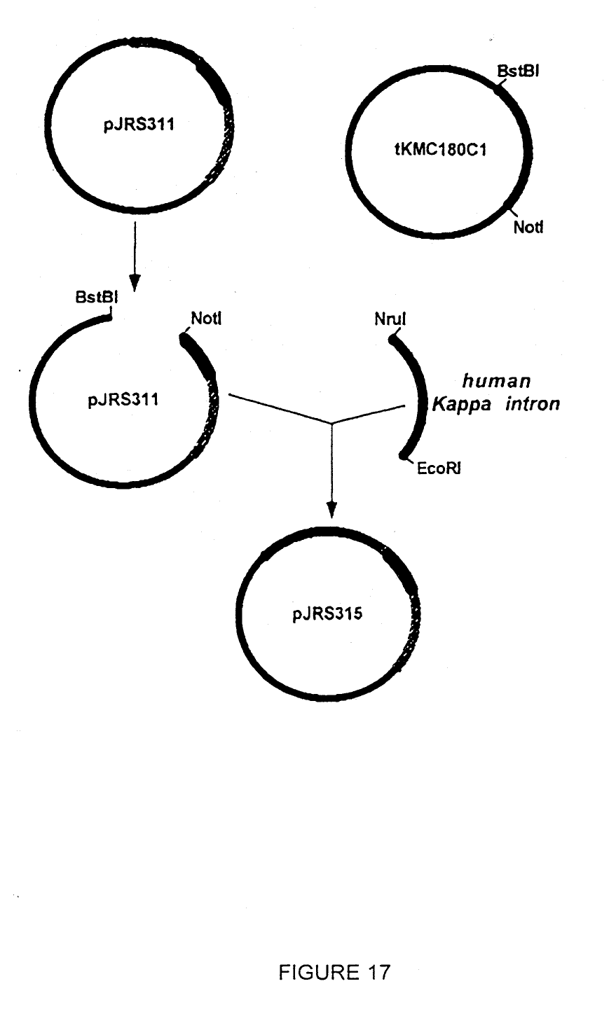

The plasmid pJRS315 is the expression plasmid into which the variable regions of the 11E10 antibody were cloned. This plasmid is a derivative of a basic expression vector, plasmid pSUN15, that contains no antibody variable region coding information, pJRS315 was created using DNA fragment ligations and site directed mutagenesis steps. The result was a vector that expresses both antibody chains with CMV promoter driven transcription (see FIG. 17). Neomycin resistance serves as the dominant selectable marker for transfection of mammalian cells. In addition, it has been designed to allow convenient cloning of any light chain variable region as an EcoRV/BstBI fragment, any heavy chain variable region as a NruI/EcoRI fragment, and any heavy chain constant domain as an EcoRI/NotI fragment. These restriction sites were chosen because they occur rarely (if ever) in human and mouse variable regions. There is a mouse J region/kappa intron fragment fused to a human kappa exon so that after post-transcriptional splicing a mouse/human chimeric kappa light chain is produced.

The backbone of the vector was the plasmid pCDNA3 (Invitrogen). This plasmid was cut with HindIII/XhoI and a “light chain polylinker” DNA fragment was inserted to create the starting “light chain vector”. This linker contained the restriction sites HindIII, KpnI, ClaI, PmlI, EcoRV, XmaI, BamHI and XhoI to facilitate subsequent cloning steps to create the plasmid pCDNA3.LCPL. A SmaI/BclI DNA fragment containing a light chain leader, anti-CKMB kappa light chain genomic fragment, and 3′ UTR was cloned into the EcoRV/BamHI sites of pCDNA3.LCPL. The mouse kappa intron, exon and 3′ UTR in this fragment was derived from LCPXK2 received from Dr. Richard Near (Near, R. I. et al., Mol. Immunol. 27:901-909, (1990)). Mutagenesis was then performed to eliminate an NruI (209), MluI (229), and BstBI (2962) and to introduce an NheI (1229) and a BamHI (1214) site to create pcDNA3mut.LCPL.LCVK (see FIG. 12).

A second “heavy chain vector” was constructed from the pcDNA3mut.LCPL.LCVK plasmid by replacing the light chain expression region (HindIII/XhoI) with a “heavy chain polylinker” consisting of restriction sites HpaI, BspEI, EcoRV, KpnI, and XhoI. This plasmid was digested with EcorRV and KpnI. A SmaI/KpnI digested DNA fragment containing a heavy chain leader and an anti-CKMB IgG2b mouse heavy chain genomic fragment was then ligated into the EcoRV/KpnI digested plasmid. A KpnI/SalI oligonucleotide fragment containing a 3′ UTR and a NotI upstream of the SalI site was subsequently cloned into the KpnI/XhoI digested plasmid (knocking out the XhoI site), to create the plasmid pCDNA3mUt.HCPL.HCV2b (see FIG. 13).

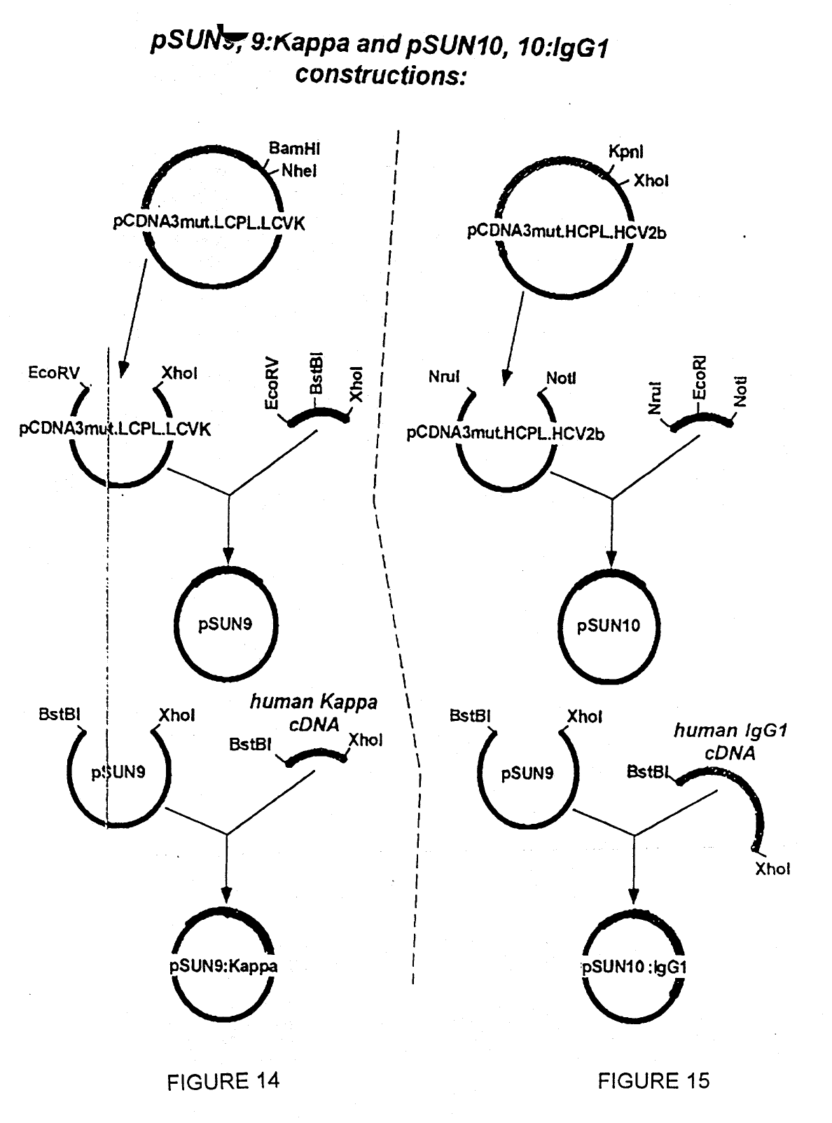

From this point, two vectors were created that did not have any of the anti-CKMB variable or constant domain DNA sequences. This was done by cutting the plasmid pcDNA3mut.LCPL.LCVK with EcoRV/XhoI and inserting a linker oligonucleotide fragment containing EcoRV, BstBI, and XhoI sites to create pSUN9 (see FIG. 14). In a similar way, the anti-CKMB fragment in pCDNA3mut.HCPL.HCV2b (NruI/NotI) was replaced by a linker oligonucleotide fragment containing NruI, EcoRI and NotI sites to create pSUN10 (see FIG. 15). A human kappa light chain constant domain was then cloned into pSUN9 as a BstBI/XhoI fragment, and a human IgG1 constant domain was cloned into pSUN10 as a EcoRI/NotI fragment.

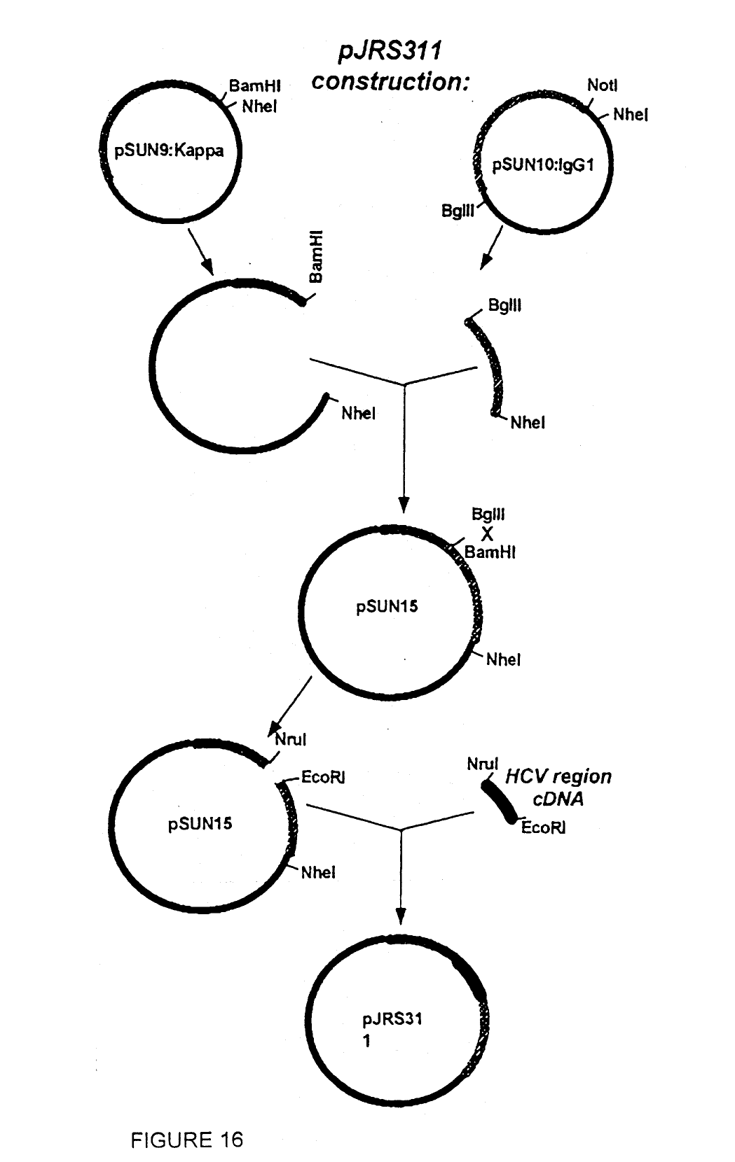

A BglII/NheI fragment from the human heavy chain vector was then cloned into the human light chain vector cut with BamHI/NheI to create pSUN15 (see FIG. 16).

The plasmid pJRS315 was then constructed using pSUN15 through the following process. A heavy chain variable region from another, unrelated, hybridoma cell line (approximately 400 bp) was digested with NruI and EcoRI (New England Biolabs), purified using a Qiaquick PCR Purification column (Qiagen), as described by the manufacturer, and ligated into NruI/EcoRI digested and gel-purified pSUN15, resulting in plasmid pJRS311 (see FIG. 16).

At this point, a BstBI/NotI (New England Biolabs) DNA fragment containing a mouse kappa J-kappa intron fragment fused to a human kappa exon fragment was digested and gel-purified from the vector tKMC180C2. This fragment was ligated into the backbone of pJRS311 digested with BstBI/NotI and gel-purified resulting in the plasmid pJRS315 (see FIG. 17).

### EXAMPLE 6

### Stable Production of Recombinant Chimeric Mouse/Human “11E10” Antibody

A. Transfection of NSO Cells

The plasmid pACE4 was transfected into NSO cells using electroporation. The plasmid was linearized with a PvuI restriction enzyme digestion. 40 μg of digested plasmid DNA was mixed with 7×10⁶ cells in a total volume of 400 μL and incubated at room temperature with gentle agitation for one minute. 10 μL of DMSO (Sigma) were added to a final concentration of 1.25%. The cells/DNA/DMSO mix was transferred to a cold 0.4 centimeter cuvette and subjected to one pulse of 250 volts, 960 μF. The cells were transferred to one well of a six-well plate containing 5 ml of non-selective media supplemented with DMSO (final concentration of 1.25%). After 24 hours at 37° C. and 10% CO₂, the cells were plated out into 96-well microtiter plates. As colonies appeared, the supernatants were assayed for the production of “humanized” antibody and for the capability for the expressed antibody to bind to the Stx2 toxin.

B. Assay for Antibody Production

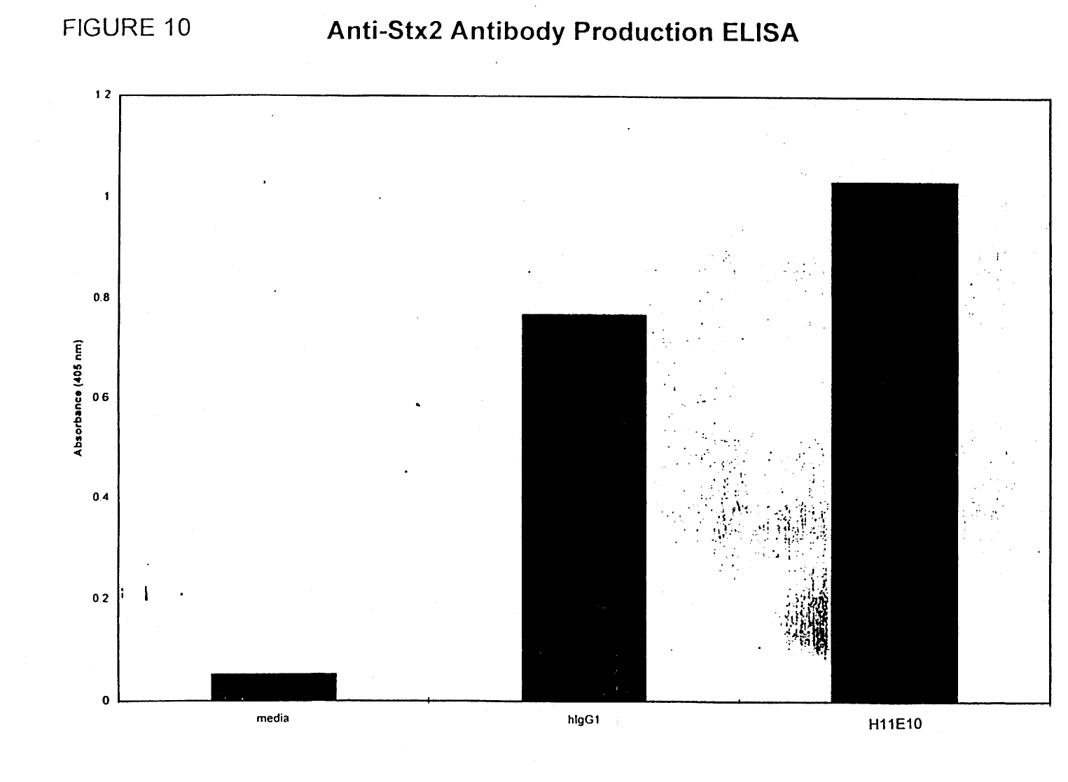

Antibody production and activity assays for the stable transfectants were performed in 8-well strips from 96-well microtiter plates (Maxisorp F8; Nunc, Inc.) coated with a 1:500 dilution of goat anti-Human F(ab′)₂anti-IgG antibody (Southern Biotechnology) using a bicarbonate coating buffer, pH 8.5. The plates were covered with pressure sensitive film (Falcon, Becton Dickinson) and incubated overnight at 4° C. Plates are then washed once with wash solution (imidazole/NaCl/0.4% Tween-20). 100 μL of culture supernatant was then applied and allowed to incubate for 30 minutes on a plate rotator at room temperature. The plates were washed five times with wash solution (imidazole/NaCl/0.4% Tween-20). A goat anti-human kappa-HRP (Southern Biotechnology) conjugate was diluted 1:800 in the sample/conjugate diluent and 100 μL was added to the samples, then incubated on a plate rotator for 30 minutes at room temperature. The samples were washed as above and then incubated with 100 μL per well of ABTS developing substrate (Kirkgaard & Perry Laboratories) and the absorbance value at 405 nm was determined using an automated microtiter plate ELISA Reader (Ceres UV900Hl, BioTek Instruments, Winooski, Vt.). This assay (see FIG. 10) demonstrates that the transfection of cells with this plasmid construct results in cells producing a molecule containing both human IgG and kappa domains.

The supernatants were then assayed for the ability of the expressed antibodies to bind to EHEC Stx2 toxin. The activity assay was performed, as above, using plates coated at 1 μg/ml with Stx2 toxin (obtained as in Example 7 from Dr. O'Brien's lab) in a bicarbonate coating buffer, pH 8.5. This assay demonstrates that the transfection of cells with this plasmid construct can result in the production of a humanized chimeric version of the 11E10 mouse monoclonal antibody which effectively binds Shiga toxin type 2 (FIG. 11).

Together, these assays demonstrate that the transfection of cells with this plasmid construct can result in the production of a stable cell line that produces a humanized chimeric version of the 11E10 mouse hybridoma antibody.

### EXAMPLE 7

### Verifying Biological and Immunological Efficacy of Humanized Antibodies to Shiga Toxin

A. Vero Cell Cytotoxicity Assay

The efficacy of the humanized antibodies to Shiga toxin obtained as detailed in Examples 1 through 6 was determined by assaying their ability to protect Vero cells from toxin. Cytotoxicity assays were performed essentially as described by Gentry and Dalrymple, J. Clin. Microbiol, 12: 361-366 (1980). Briefly, toxin was obtained from cultures of E. coli K-12 strains that contained either plasmid pLPSH3 (encodes Stx; J. Infect. Disease 164:344-352 (1991)) or pMJ 100 (encodes Stx2; Inf. and Immunity, 57:3743-3750 (1989)). Bacteria were disrupted by sonic lysis and clarified by centrifugation. The extracts were serially diluted in tissue culture medium (Dulbecco modified Eagle medium containing 10% fetal calf serum, 0.8 mM glutamine, 500 U of penicillin G per ml, and 500 mg of streptomycin per ml). One hundred microliters of 10-fold dilutions of the lysates were added to microtiter plate wells containing about 10⁴ Vero cells in 100 μl of medium. The tissue culture cells were incubated at 37° C. in 5% CO₂ for 48 hours and then fixed and stained with crystal violet. The intensity of color of the fixed and stained cells was measured with a Titertek reader at 620 nm. Incubation without antibody provides a standard toxicity curve for the Stx.

B. Antisera Neutralization Assay

Humanized mouse antibodies obtained according to the methods described in Examples 1 through 6 were tested for toxin neutralization. Neutralization of cytotoxic activity was described in great detail in Schmitt et al., Infect. and Immun., 59:1065-1073 (1991). Briefly, lysates were incubated with serial dilutions of the humanized mouse antibodies at 37° C. for 2 hours. One hundred microliters of the samples were then added to Vero cells as described above.

TABLE 1
Vero Cell Neutralization Assay

| Antibody | Required ng to Neut. 1 CD₅₀* |
|---|---|
| Mouse 13C4 | 25 |
| Humanized 13C4 | 26 |
| Mouse 11E10 | 2.7 |
| Humanized 11E10 | 82.8 |

*(1 CD₅₀ = 1 pg pure toxin)

### EXAMPLE 8

### Passive Immunization with Humanized Antibodies

A. Protection Against Injection With Crude Stx1 Toxin.

At day zero, groups of five CD-1 mice were injected intraperitoneally (0.1 ml) with antibody H13C4 (humanized α-Stx1B), 13C4 (mouse α-Stx1B), H11E10 (humanized α-Stx2A), or phosphate buffered saline (PBS). These injections were repeated at day 1. The mice were then challenged by intravenous injection with crude Stx1 toxin (obtained as described in Example 7) at doses of 1.7×10⁵ or 1.7×10⁶ CD₅₀. These doses of toxin were chosen following preliminary experiments with varying amounts of toxin. Mice were monitored for 21 days.

The results (Table 2) show clearly that the injected antibodies protect the mice against at least 10 times the normal lethal toxin dose. As a negative control, the antibodies to Stx2 did not protect against Stx1 toxin challenge, indicating that the protection was specific to the toxin/antibody pair, and was not an artifact of the antibody preparation process.

TABLE 2
CD-1 Mice Injected Intravenously with Crude Stx1

| | | μg/kg |
|---|---|---|
| | #LD₅₀ Protected | of Ab to Protect from |
| Antibody (dose/mouse) | Against | 1LD₅₀* |
| Murine13C4 (1.4 μg) | 10 | 6.1 |
| Humanized 13C4 (4.1 μg) | 20 | 8.9 |
| Humanized 11E10 (232 μg) | 0 | No protection |

*(1 LD₅₀ = 30 ng crude Stx1)

B. Protection Against Oral Infection With Stx2-Producing EHEC Strains.

Two different strains of mice and bacteria were used in these studies to test efficacy against both Stx2 and Stx2-variant. DBA/2J mice were challenged with EHEC strain 86-24 (O157:H7, Stx2⁺) and CD-1 mice were challenged with strain B2F1 (091:H21, Stx2-variant⁺). While E. coli strain B2F1 is normally fatal to both mice strains, E. coli strain 86-24 is fatal to DBA/2J mice, while CD-1 mice will survive 86-24 infection.

At day zero, antibody H11E10 (humanized α-Stx2A) or 11E10 (mouse α-Stx2A) was injected intraperitoneally (0.1 ml) into groups of four or five mice. Control groups included mice that had received antibody 13C4 (mouse α-Stx1B), mice that had received 11E10 ascites fluid (mouse α-Stx2A), or mice that had received PBS instead of antibody. Mice were given streptomycin (5 g/L) in their drinking water to decrease normal intestinal flora and their food was removed. Streptomycin resistant derivatives of strains 86-24 (O157:H7, Stx2⁺) and B2F1 (O91:H21, Stx2-variant⁺) were grown overnight in L broth.

The following day (day 1) the mice received a second injection of test antibody, control antibody, or PBS. The mice were immediately fed 10¹⁰ CFU of 86-24 that had been pelleted and resuspended in 20% sucrose or 10³ CFU of B2F1 that had been serially diluted in 20% sucrose. Food was returned to the cage and the mice were monitored for 21 days. (CFU=Colony Forming Units) As shown in Table 3, immunization of the mice with the either the murine or the humanized anti-Stx2 antibodies resulted in complete protection from a lethal oral dose of EHEC. Immunization with mouse 13C4 antibody, prepared in the same way but immunoreactive with the Stx1 toxin instead of the Stx2 toxin did not protect the mice from challenge with 86-24, a result that indicates the immunospecificity of the response.

TABLE 3
DBA/2J Mice Fed 10¹⁰ CFU 0157 (Stx2)

| Antibody (dose/mouse) | Survivors |
|---|---|
| Murine 13C4 (1.4 μg) | 0/5 |
| Murine 11E10 (1.0 μg) | 5/5 |
| Humanized 11E10 (1.0 μg) | 5/5 |

Similar results are illustrated in Table 4, where the more resistant CD-1 mice were fed the B2F 1 E. coli strain which produces Stx2-variant toxin. Without treatment with antibody, mortality was total, but both the mouse and the humanized antibodies protected against Stx2-variant in a dose dependent manner.

TABLE 4
CD-1 Mice Fed 10³ CFU O91: H21 (Stx2-variant)

| Antibody (dose/mouse) | Survivors |
|---|---|
| PBS | 0/5 |
| Murine 11E10 (8.7 μg) | 1/4* |
| Murine 11E10 (6.45 mg) | 5/5 |
| Humanized 11E10 (23.2 μg) | 0/5 |
| Humanized 11E10 (232 μg) | 5/5 |

Protective dose between 10.1 and 1.0 mg/kg
*Delayed mean time to death by 3.4 days

### EXAMPLE 9

### Treatment of Disease Caused by Bacteria Producing Shiga Toxin

The subject invention also provides for a variety of methods of treating, ameliorating, or preventing, the diseases and effects associated with exposure to Shiga toxin. Positive clinical responses in humans have been obtained with monoclonal antibodies, and one skilled in the art would know how to employ anti-Stx humanized monoclonal antibodies in humans. (See Fagerberg et al., Cancer Research, 55:1824-27 (1995); Eur. J. Cancer, 2:261-267 (1995)). The precise dosage of humanized anti-Shiga toxin antibody administered to a patient for treatment of these diseases will vary in accordance with factors appreciated by the typical clinician. These factors include (but are not limited to) size, age, overall health, the extent of infection, and other medications being administered to said patient. The development of a precise treatment regimen will require optimization through routine medical procedures well known to those in the medical arts. Examples of potential patient groups would include (but not be limited to) young children with bloody diarrhea but no white cells in the stool, patients with indications of HUS, patients with positive stool sample tests for Shiga toxin, siblings or daycare cohorts in contact with a case (as a passive preventative measure), and any patient with diarrhea (not necessarily bloody) that has been in contact with an identified case. A typical dosage of about 5 mg/kg body weight of humanized 13C4 combined with about 10 mg/kg body weight of humanized 11E10 would be contemplated. This combined formulation could be administered to the patient twice to ensure effectiveness. Inclusion of both types of humanized antibodies together provides assurance that the patient will be protected against all types of Shiga toxin.

The person skilled in the art would understand how to use and practice the invention based on the above disclosure. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

We claim:

1. A humanize monoclonal antibody that binds to Shiga toxin protein, comprising a constant region and a variable region, wherein said constant region contains at least part of a human immunoglobulin constant region and said variable region contains at least part of a non-human immunoglobulin variable region.

2. The humanized monoclonal antibody of claim 1, having the same binding specificity as the antibody selected from the group consisting of murine 13C4 (ATCC Accession No. CRL 1794), murine 11E10 (ATCC Accession No. CRL 1987), humanized 13C4 (H13C4), and humanized 11E10 (H11E10).

3. The humanized monoclonal antibody of claim 1, wherein the antibody binds Shiga toxin type 1.

4. The humanized monoclonal antibody of claim 3, wherein said non-human variable region is from the mouse.

5. The humanized monoclonal antibody of claim 3, wherein at least part of said variable region is from the sequences as set forth in FIG. 3 (SEQ ID NO:19 and SEQ ID NO:21).

6. A fragment of the antibody of claim 3, wherein the fragment binds Shiga toxin type 1.

7. The humanized monoclonal antibody of claim 3, wherein said human constant region is selected from the group consisting of IgG, IgA and IgM.

8. The humanized monoclonal antibody of claim 7, wherein said human constant region is IgG.

9. A humanized monoclonal antibody which binds Shiga toxin type 1, comprising a constant region and a variable region, wherein:
said constant region is IgG1-kappa, and
said variable region contains at least part of the sequence as set forth in FIG. 3 (SEQ ID NO: 19 and SEQ ID NO: 21).

10. A humanized monoclonal antibody which binds Shiga toxin type 1, comprising a constant region and a variable region, wherein:
said constant region is IgG1-kappa, and said variable region contains at least part of the CDR sequences as set forth in FIG. 3, said CDR sequences located as follows:

| | |
|---|---|
| Heavy Chain CDRs: | CDR1-aa31-35 |
| (SEQ ID NO: 19) | CDR2-aa50-66 |
| | CDR3-aa99-111 |
| Light Chain CDRs: | CDR1-aa24-34 |
| (SEQ ID NO: 21) | CDR2-aa50-56 |
| | CDR3-aa89-97 |

11. An expression vector comprising a DNA sequence encoding the variable and constant regions of the light and the heavy chains of the antibody of claim 9, where the coding sequences for the variable regions contain at least part of the DNA sequences as set forth in FIG. 3 (SEQ ID NO:18 and SEQ ID NO:20).

12. A host cell transformed with the expression vector of claim 11.

13. The humanized monoclonal antibody of claim 1, wherein the antibody binds Shiga toxin type 2 and Shiga toxin type 2 variants.

14. The humanized monoclonal antibody of claim 13, wherein said non-human variable region is from the mouse.

16. The humanized monoclonal antibody of claim 13, wherein at least part of said variable region is from the sequence as set forth in FIG. 6 (SEQ ID NO: 42 and SEQ ID NO:44).

15. A fragment of the antibody of claim 13, wherein the fragment binds Shiga toxin type 2 and Shiga toxin type 2 variants.

16. The humanized monoclonal antibody of claim 13, wherein said human constant region is selected from group consisting of IgG, IgA and IgM.

17. The humanized monoclonal antibody of claim 17, wherein said human constant region is IgG.

18. A humanized monoclonal antibody which binds Shiga toxin type 2 and Shiga toxin type 2 variants, comprising a constant region and a variable region, wherein:
said constant region is IgG1-kappa, and
said variable region contains at least part of the sequence as set forth in FIG. 6 (SEQ ID NO:42 and SEQ ID NO:44).

19. A humanized monoclonal antibody which binds Shiga toxin type 2 and Shiga toxin type 2 variants, comprising a constant region and a variable region, wherein:
said constant region is IgG1-kappa, and
said variable region contains at least part of the CDR sequences as set forth in FIG. 6, said CDR sequences located follows:

| | |
|---|---|
| Heavy Chain CDRs: | CDR1-aa31-35 |
| (SEQ ID NO: 44) | CDR2-aa50-66 |
| | CDR3-aa99-108 |
| Light Chain CDRs: | CDR1-aa24-40 |
| (SEQ ID NO: 42) | CDR2-aa56-62 |
| | CDR3-aa95-103 |

20. An expression vector comprising a DNA encoding the variable and constant regions of the light and the heavy chains of the antibody of claim 19, where the coding sequences for the variable regions contain at least part of the DNA sequences as set forth in FIG. 6 (SEQ ID NO:41 and 43)

21. A host cell transformed with the expression vector of claim 20.

22. A pharmaceutical composition comprising the antibody of claim 1, or fragment or derivative thereof, and a pharmaceutically acceptable carrier or diluent.

23. A method for treating a patient having an infection caused by EHEC or other Shiga toxin producing bacteria comprising administering to the patient a therapeutically effective amount of the pharmaceutical composition of claim 22.

24. A method for reducing illness caused by EHEC or other Shiga toxin-producing bacteria comprising administering to the patient a prophylactically effective amount of the pharmaceutical composition of claim 22.

25. A pharmaceutical composition comprising the antibody of claim 3, or fragment or derivative thereof, and a pharmaceutically acceptable carrier or diluent.

26. A method for treating a patient having an infection caused by EHEC or other Shiga toxin producing bacteria comprising administering to the patient a therapeutically effective amount of the pharmaceutical composition of claim 25.

27. A method for reducing illness caused by EHEC or other Shiga toxin-producing bacteria comprising administering to the patient a prophylactically effective amount of the pharmaceutical composition of claim 25.

28. A pharmaceutical composition comprising the antibody of claim 13, or fragment or derivative thereof, and a pharmaceutically acceptable carrier or diluent.

29. A method for treating a patient having an infection caused by EHEC or other Shiga toxin producing bacteria comprising administering to the patient a therapeutically effective amount of the pharmaceutical composition of claim 28.

30. A method for reducing illness caused by EHEC or other Shiga toxin-producing bacteria comprising administering to the patient a prophylactically effective amount of the pharmaceutical composition of claim 28.


SEQUENCE LISTING (44 sequences)

SEQ ID NO: 1 - DNA, 45 bp, Artificial Sequence
  Description of Artificial Sequence Synthetic Oligonucleotide
atttcaggcccagccggccatggccgargtrmagctksakgagwc

SEQ ID NO: 2 - DNA, 45 bp, Artificial Sequence
  Description of Artificial Sequence Synthetic Oligonucleotide
atttcaggcccagccggccatggccgargtycarctkcarcaryc

SEQ ID NO: 3 - DNA, 45 bp, Artificial Sequence
  Description of Artificial Sequence Synthetic Oligonucleotide
atttcaggcccagccggccatggcccaggtgaagctkstsgartc

SEQ ID NO: 4 - DNA, 45 bp, Artificial Sequence; identical to SEQ ID NO: 1
  Description of Artificial Sequence Synthetic Oligonucleotide

SEQ ID NO: 5 - DNA, 45 bp, Artificial Sequence
  Description of Artificial Sequence Synthetic Oligonucleotide
atttcaggcccagccggccatggcccaggtbcarctkmarsartc

SEQ ID NO: 6 - DNA, 20 bp, Artificial Sequence
  Description of Artificial Sequence Synthetic Oligonucleotide
gaartavcccttgaccaggc

SEQ ID NO: 7 - DNA, 35 bp, Artificial Sequence
  Description of Artificial Sequence Synthetic Oligonucleotide
ggaggcggcggttctgacattgtgmtgwcmcartc

SEQ ID NO: 8 - DNA, 35 bp, Artificial Sequence
  Description of Artificial Sequence Synthetic Oligonucleotide
ggaggcggcggttctgatrttkygatgacbcarrc

SEQ ID NO: 9 - DNA, 35 bp, Artificial Sequence
  Description of Artificial Sequence Synthetic Oligonucleotide
ggaggcggcggttctgayatymagatgacmcagwc

SEQ ID NO: 10 - DNA, 35 bp, Artificial Sequence
  Description of Artificial Sequence Synthetic Oligonucleotide
ggaggcggcggttctsaaattgwkctsacycagtc

SEQ ID NO: 11 - DNA, 40 bp, Artificial Sequence
  Description of Artificial Sequence Synthetic Oligonucleotide
ttcataggcggccgcactagtagcmcgtttcagytccarc

SEQ ID NO: 12 - DNA, 40 bp, Artificial Sequence
  Description of Artificial Sequence Synthetic Oligonucleotide
ttcataggcggccgcactagtagcmcgtttkatytccarc

SEQ ID NO: 13 - DNA, 23 bp, Artificial Sequence
  Description of Artificial Sequence Synthetic Oligonucleotide
gcacctccagatgttaactgctc

SEQ ID NO: 14 - DNA, 49 bp, Artificial Sequence
  Description of Artificial Sequence Synthetic Oligonucleotide
cttgatcgcgacagctacaggtgtccactcccaggtgcagctgcaggag

SEQ ID NO: 15 - DNA, 36 bp, Artificial Sequence
  Description of Artificial Sequence Synthetic Oligonucleotide
ggtatggaattctgaggagactgtgagagtggtgcc

SEQ ID NO: 16 - DNA, 36 bp, Artificial Sequence
  Description of Artificial Sequence Synthetic Oligonucleotide
ggttctgatatcgtgatgtcccagtctcacaaattc

SEQ ID NO: 17 - DNA, 42 bp, Artificial Sequence
  Description of Artificial Sequence Synthetic Oligonucleotide
gacatattcgaaaagtgtacttacgtttcagctccagactgg

SEQ ID NO: 18 - DNA, 366 bp, Shigella dysenteriae
caggtgcagctgcaggagtctggggctgagctggtgaggtctggggcctcagtgaggatgtcctgcaaggcttctggctacacatttaccagttacaatatgcactgggtaaaacagacacctggacagggcctggaatggattggatatatttatcctggaaatggtggtactaactacattcagaaatttaagggcaaggccatattgactgcagacacatcctccagcacagcctacatgcagatcagcagtctgacatctgaagactctgcggtctatttctgtacaagaagtccctctcactacagtagtgacccctactttgactactggggccagggcaccactctcacagtctcctca

SEQ ID NO: 19 - protein, 122 aa, Shigella dysenteriae
QVQLQESGAELVRSGASVRMSCDASGYTFTSYNMHWVKQTPGQGLEWIGYIYPGNGGTNYIQKFKGKAILTADTSSSTAYMQISSLTSEDSAVYFCTRSPSHYSSDPYFDYWGQGTTLTVSS

SEQ ID NO: 20 - DNA, 324 bp, Shigella dysenteriae
gatatcgtgatgtcccagtctcacaaattcatgtccacatcagtcggagacagggtcagcatcacctgtaaggccagccaggatgtgggtactgctgttgcctggtatcagcagaatccaggacaatctcctaaatttctgatttactgggcatccacacggcacactggagtccctgatcgcttcacaggcagtggatctgggacagatttcactctcaccattaccaatgtgcagtctgaagacttggcagattatttctgtcagcaatatagcagttatcctctcacgttcggtgctgggaccagtctggagctgaaacgt

SEQ ID NO: 21 - protein, 108 aa, Shigella dysenteriae
DIVMSQSHKFMSTSVGDRVSITCKASQDVGTAVAWYQQNPGQSPKFLIYWASTRHTGVPDRFTGSGSGTDFTLTITNVQSEDLADYFCQQYSSYPLTFGAGTSLELKR

SEQ ID NO: 22 - DNA, 45 bp, Artificial Sequence; identical to SEQ ID NO: 1
  Description of Artificial Sequence Synthetic Oligonucleotide

SEQ ID NO: 23 - DNA, 45 bp, Artificial Sequence; identical to SEQ ID NO: 2
  Description of Artificial Sequence Synthetic Oligonucleotide

SEQ ID NO: 24 - DNA, 45 bp, Artificial Sequence; identical to SEQ ID NO: 3
  Description of Artificial Sequence Synthetic Oligonucleotide

SEQ ID NO: 25 - DNA, 45 bp, Artificial Sequence
  Description of Artificial Sequence Synthetic Oligonucleotide
atttcaggcccagccggccatggccgavgtgmwgctkgtggagwc

SEQ ID NO: 26 - DNA, 45 bp, Artificial Sequence; identical to SEQ ID NO: 5
  Description of Artificial Sequence Synthetic Oligonucleotide

SEQ ID NO: 27 - DNA, 20 bp, Artificial Sequence
  Description of Artificial Sequence Synthetic Oligonucleotide
gaartavcccttgaccaggc

SEQ ID NO: 28 - DNA, 35 bp, Artificial Sequence
  Description of Artificial Sequence Synthetic Oligonucleotide
gctgccaccgccacctgmrgagacdgtgastgarg

SEQ ID NO: 29 - DNA, 35 bp, Artificial Sequence
  Description of Artificial Sequence Synthetic Oligonucleotide
gctgccaccgccacctgmrgagacdgtgasmgtrg

SEQ ID NO: 30 - DNA, 35 bp, Artificial Sequence
  Description of Artificial Sequence Synthetic Oligonucleotide
gctgccaccgccacctgmrgagacdgtgascagrg

SEQ ID NO: 31 - DNA, 35 bp, Artificial Sequence; identical to SEQ ID NO: 7
  Description of Artificial Sequence Synthetic Oligonucleotide

SEQ ID NO: 32 - DNA, 35 bp, Artificial Sequence; identical to SEQ ID NO: 8
  Description of Artificial Sequence Synthetic Oligonucleotide

SEQ ID NO: 33 - DNA, 35 bp, Artificial Sequence; identical to SEQ ID NO: 9
  Description of Artificial Sequence Synthetic Oligonucleotide

SEQ ID NO: 34 - DNA, 35 bp, Artificial Sequence; identical to SEQ ID NO: 10
  Description of Artificial Sequence Synthetic Oligonucleotide

SEQ ID NO: 35 - DNA, 40 bp, Artificial Sequence; identical to SEQ ID NO: 11
  Description of Artificial Sequence Synthetic Oligonucleotide

SEQ ID NO: 36 - DNA, 40 bp, Artificial Sequence; identical to SEQ ID NO: 12
  Description of Artificial Sequence Synthetic Oligonucleotide

SEQ ID NO: 37 - DNA, 52 bp, Artificial Sequence
  Description of Artificial Sequence Synthetic Oligonucleotide
atatactcgcgacagctacaggtgtccactccgaagtccaactgcaacagcc

SEQ ID NO: 38 - DNA, 34 bp, Artificial Sequence
  Description of Artificial Sequence Synthetic Oligonucleotide
attaatgaattctgcggagacggtgagagtggtc

SEQ ID NO: 39 - DNA, 29 bp, Artificial Sequence
  Description of Artificial Sequence Synthetic Oligonucleotide
ttaaatgatatcgtgctgtcacaatctcc

SEQ ID NO: 40 - DNA, 45 bp, Artificial Sequence
  Description of Artificial Sequence Synthetic Oligonucleotide
taatcgttcgaaaagtgtacttacgtttcagttccagcttggtcc

SEQ ID NO: 41 - DNA, 339 bp, Shigella dysenteriae
gacattgtgctgtcacaatctccatcctccctagttgtgtcagttggagagaaggttactatgagctgcaagtctagtcagagccttttatatagtagaaatcaaaagaactacttggcctggtaccagcagaaaccagggcagtctcctaaagtgctgatttactgggcatctactagggaatctggggtccctgatcgcctcacaggcagtggatctgggacagatttcactctcaccatcagcagtgtgaaggctgaagacctggcagtttattactgtcagcaatattatagttatccgctcacgttcggtgctgggaccaagctggagctgaaa

SEQ ID NO: 42 - protein, 113 aa, Shigella dysenteriae
DIVLSQSPSSLVVSVGEKVTMSCKSSQSLLYSRNQKNYLAWYQQKPGQSPKVLIYWASTRESGVPDRLTGSGSGTDFTLTISSVKAEDLAVYYCQQYYSYPLTFGAGTKLELK

SEQ ID NO: 43 - DNA, 357 bp, Shigella dysenteriae
gaagtccaactgcaacagcctggacctgagctggagaagcctggcgcttcagtgaaactatcctgcaaggcttctggttactctttcactgactacaacatgaactgggtgaagcagaacaatggagagagccttgagtggattggaaaaattgatccttactatggtggtcctagctacaaccagaagttcaaggacaaggccacattgactgtagacaagtcttccagcacagcctacatgcagttcaagagcctgacatctgaggactctgcagtctattactgtacaagagggggaaatagggactggtacttcgatgtgtggggcgcagggaccacgctcaccgtctccgca

SEQ ID NO: 44 - protein, 119 aa, Shigella dysenteriae
EVQLQQPGPELEKPGASVKLSCKASGYSFTDYNMNWVKQNNGESLEWIGKIDPYYGGPSYNQKFKDKATLTVDKSSSTAYMQFKSLTSEDSAVYYCTRGGNRDWYFDVWGAGTTLTVSA